SEQUENCE LISTING (85 sequences)

SEQ ID NO: 1 - DNA, 1344 bp, Artificial Sequence
  Synthesized
caggtgcagctgcaggagagcggccccggcctggtgaagcccagcgagaccctgagcctgacctgcaccgtgagcggcggcagcgtgagcagcggcgactactactggacctggatccgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 2 - protein, 448 aa, Artificial Sequence
  Synthesized
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - DNA, 642 bp, Artificial Sequence
  Synthesized
gacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaagcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgt

SEQ ID NO: 4 - protein, 214 aa, Artificial Sequence
  Synthesized
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 1836 bp, Artificial Sequence
  Synthesized
ctggacagcccagataggccatggaacccacctactttctctcctgcactgctggtggttacagaaggagataatgctacctttacttgctccttttccaacactagtgagagttttgtccttaattggtatagaatgtctccttcaaatcagacggacaagctcgctgcatttcctgaggaccgcagtcagccggggcaagattgcagattccgcgtgacccagctccccaacggacgcgattttcacatgtccgttgtcagggcacgacgcaacgatagtgggacttatctgtgcggggcgatcagtctggccccgaaggcccagataaaagagtccctccgcgctgaactcagggtgaccgagagacgggccgaagtgcccacagcacacccaagtccaagcccaagacctgctgggcaattccaagggggtggcgaagcagctgctaaggaggcagccgcaaaggaagcagctgcaaaggcaggaggccaggtgcagctgcaggagagcggccccggcctggtgaagcccagcgagaccctgagcctgacctgcaccgtgagcggcggcagcgtgagcagcggcgactactactggacctggatccgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 6 - protein, 612 aa, Artificial Sequence
  Synthesized
LDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQGGGEAAAKEAAAKEAAAKAGGQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - DNA, 1899 bp, Artificial Sequence
  Synthesized
ctggacagcccagataggccatggaacccacctactttctctcctgcactgctggtggttacagaaggagataatgctacctttacttgctccttttccaacactagtgagagttttgtccttaattggtatagaatgtctccttcaaatcagacggacaagctcgctgcatttcctgaggaccgcagtcagccggggcaagattgcagattccgcgtgacccagctccccaacggacgcgattttcacatgtccgttgtcagggcacgacgcaacgatagtgggacttatctgtgcggggcgatcagtctggccccgaaggcccagataaaagagtccctccgcgctgaactcagggtgaccgagagacgggccgaagtgcccacagcacacccaagtccaagcccaagacctgctgggcaattccaaggcggtagtggaagaggtgcagctcctgctgcagcacccgcaaaacaagaagcagcggctcccgctcccgccgcaaaagcagaagccccagctgccgcgcccgctgctaaggctggaggttccggacaggtgcagctgcaggagagcggccccggcctggtgaagcccagcgagaccctgagcctgacctgcaccgtgagcggcggcagcgtgagcagcggcgactactactggacctggatccgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 8 - protein, 633 aa, Artificial Sequence
  Synthesized
LDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQGGSGRGAAPAAAPAKQEAAAPAPAAKAEAPAAAPAAKAGGSGQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - DNA, 1134 bp, Artificial Sequence
  Synthesized
ctggacagcccagataggccatggaacccacctactttctctcctgcactgctggtggttacagaaggagataatgctacctttacttgctccttttccaacactagtgagagttttgtccttaattggtatagaatgtctccttcaaatcagacggacaagctcgctgcatttcctgaggaccgcagtcagccggggcaagattgcagattccgcgtgacccagctccccaacggacgcgattttcacatgtccgttgtcagggcacgacgcaacgatagtgggacttatctgtgcggggcgatcagtctggccccgaaggcccagataaaagagtccctccgcgctgaactcagggtgaccgagagacgggccgaagtgcccacagcacacccaagtccaagcccaagacctgctgggcaattccaagggggtggcgaagcagctgctaaggaggcagccgcaaaggaagcagctgcaaaggcaggaggcgacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaagcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgt

SEQ ID NO: 10 - protein, 378 aa, Artificial Sequence
  Synthesized
LDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQGGGEAAAKEAAAKEAAAKAGGDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 1197 bp, Artificial Sequence
  Synthesized
ctggacagcccagataggccatggaacccacctactttctctcctgcactgctggtggttacagaaggagataatgctacctttacttgctccttttccaacactagtgagagttttgtccttaattggtatagaatgtctccttcaaatcagacggacaagctcgctgcatttcctgaggaccgcagtcagccggggcaagattgcagattccgcgtgacccagctccccaacggacgcgattttcacatgtccgttgtcagggcacgacgcaacgatagtgggacttatctgtgcggggcgatcagtctggccccgaaggcccagataaaagagtccctccgcgctgaactcagggtgaccgagagacgggccgaagtgcccacagcacacccaagtccaagcccaagacctgctgggcaattccaaggcggtagtggaagaggtgcagctcctgctgcagcacccgcaaaacaagaagcagcggctcccgctcccgccgcaaaagcagaagccccagctgccgcgcccgctgctaaggctggaggttccggagacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaagcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgt

SEQ ID NO: 12 - protein, 399 aa, Artificial Sequence
  Synthesized
LDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQGGSGRGAAPAAAPAKQEAAAPAPAAKAEAPAAAPAAKAGGSGDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - DNA, 1836 bp, Artificial Sequence
  Synthesized
ctggacagcccagataggccatggaacccacctactttctctcctgcactgctggtggttacagaaggagataatgctacctttacttgctccttttccaacactagtgagagttttcacgtggtgtggcacagagagtctccttcaggccagacggacaccctcgctgcatttcctgaggaccgcagtcagccggggcaagattgcagattccgcgtgacccagctccccaacggacgcgattttcacatgtccgttgtcagggcacgacgcaacgatagtgggacttatgtgtgcggggtgatcagtctggccccgaagatccagataaaagagtccctccgcgctgaactcagggtgaccgagagacgggccgaagtgcccacagcacacccaagtccaagcccaagacctgctgggcaattccaagggggtggcgaagcagctgctaaggaggcagccgcaaaggaagcagctgcaaaggcaggaggccaggtgcagctgcaggagagcggccccggcctggtgaagcccagcgagaccctgagcctgacctgcaccgtgagcggcggcagcgtgagcagcggcgactactactggacctggatccgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 14 - protein, 612 aa, Artificial Sequence
  Synthesized
LDSPDRPWNPPTFSPALLVVTEGDNATFTCSFSNTSESFHVVWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQGGGEAAAKEAAAKEAAAKAGGQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - DNA, 1899 bp, Artificial Sequence
  Synthesized
ctggacagcccagataggccatggaacccacctactttctctcctgcactgctggtggttacagaaggagataatgctacctttacttgctccttttccaacactagtgagagttttcacgtggtgtggcacagagagtctccttcaggccagacggacaccctcgctgcatttcctgaggaccgcagtcagccggggcaagattgcagattccgcgtgacccagctccccaacggacgcgattttcacatgtccgttgtcagggcacgacgcaacgatagtgggacttatgtgtgcggggtgatcagtctggccccgaagatccagataaaagagtccctccgcgctgaactcagggtgaccgagagacgggccgaagtgcccacagcacacccaagtccaagcccaagacctgctgggcaattccaaggcggtagtggaagaggtgcagctcctgctgcagcacccgcaaaacaagaagcagcggctcccgctcccgccgcaaaagcagaagccccagctgccgcgcccgctgctaaggctggaggttccggacaggtgcagctgcaggagagcggccccggcctggtgaagcccagcgagaccctgagcctgacctgcaccgtgagcggcggcagcgtgagcagcggcgactactactggacctggatccgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 16 - protein, 633 aa, Artificial Sequence
  Synthesized
LDSPDRPWNPPTFSPALLVVTEGDNATFTCSFSNTSESFHVVWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQGGSGRGAAPAAAPAKQEAAAPAPAAKAEAPAAAPAAKAGGSGQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - DNA, 1134 bp, Artificial Sequence
  Synthesized
ctggacagcccagataggccatggaacccacctactttctctcctgcactgctggtggttacagaaggagataatgctacctttacttgctccttttccaacactagtgagagttttcacgtggtgtggcacagagagtctccttcaggccagacggacaccctcgctgcatttcctgaggaccgcagtcagccggggcaagattgcagattccgcgtgacccagctccccaacggacgcgattttcacatgtccgttgtcagggcacgacgcaacgatagtgggacttatgtgtgcggggtgatcagtctggccccgaagatccagataaaagagtccctccgcgctgaactcagggtgaccgagagacgggccgaagtgcccacagcacacccaagtccaagcccaagacctgctgggcaattccaagggggtggcgaagcagctgctaaggaggcagccgcaaaggaagcagctgcaaaggcaggaggcgacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaagcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgt

SEQ ID NO: 18 - protein, 378 aa, Artificial Sequence
  Synthesized
LDSPDRPWNPPTFSPALLVVTEGDNATFTCSFSNTSESFHVVWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQGGGEAAAKEAAAKEAAAKAGGDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - DNA, 1197 bp, Artificial Sequence
  Synthesized
ctggacagcccagataggccatggaacccacctactttctctcctgcactgctggtggttacagaaggagataatgctacctttacttgctccttttccaacactagtgagagttttcacgtggtgtggcacagagagtctccttcaggccagacggacaccctcgctgcatttcctgaggaccgcagtcagccggggcaagattgcagattccgcgtgacccagctccccaacggacgcgattttcacatgtccgttgtcagggcacgacgcaacgatagtgggacttatgtgtgcggggtgatcagtctggccccgaagatccagataaaagagtccctccgcgctgaactcagggtgaccgagagacgggccgaagtgcccacagcacacccaagtccaagcccaagacctgctgggcaattccaaggcggtagtggaagaggtgcagctcctgctgcagcacccgcaaaacaagaagcagcggctcccgctcccgccgcaaaagcagaagccccagctgccgcgcccgctgctaaggctggaggttccggagacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaagcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgt

SEQ ID NO: 20 - protein, 399 aa, Artificial Sequence
  Synthesized
LDSPDRPWNPPTFSPALLVVTEGDNATFTCSFSNTSESFHVVWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQGGSGRGAAPAAAPAKQEAAAPAPAAKAEAPAAAPAAKAGGSGDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - DNA, 2127 bp, Artificial Sequence
  Synthesized
gaggtgcagctggtggagagcggaggtggactagtacagcctggtggcagcctacgactgagttgcgccgccagcggcttcaccttcagcgacagctggatacactgggtgcgccaggcccccggcaagggcctggagtgggtggcctggatcagcccctacggcggcagcacctactacgccgacagcgtgaagggccgcttcaccatcagcgccgacaccagcaagaacaccgcctacctgcagatgaacagcctgcgcgccgaggacaccgccgtgtactactgcgcccgccgccactggcccggcggcttcgactactggggccagggcaccctggtgaccgtgagcagcggaggcgggggaagcggcggcggagggtctggaggagggggcagtgacatccagatgacccagagccccagcagcctgagcgccagcgtgggcgaccgcgtgaccatcacctgccgcgccagccaggacgtgagcaccgccgtggcctggtaccagcagaagcccggcaaggcccccaagctgctgatctacagcgccagcttcctgtacagcggcgtgcccagccgcttcagcggcagcggcagcggcaccgacttcaccctgaccatcagcagcctgcagcccgaggacttcgccacctactactgccagcagtacctgtaccaccccgccaccttcggccagggcaccaaggtggagatcaaggggggtggcgaagcagctgctaaggaggcagccgcaaaggaagcagctgcaaaggcaggaggccaggtgcagctgcaggagagcggccccggcctggtgaagcccagcgagaccctgagcctgacctgcaccgtgagcggcggcagcgtgagcagcggcgactactactggacctggatccgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 22 - protein, 709 aa, Artificial Sequence
  Synthesized
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGGEAAAKEAAAKEAAAKAGGQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - DNA, 2190 bp, Artificial Sequence
  Synthesized
gaggtgcagctggtggagagcggaggtggactagtacagcctggtggcagcctacgactgagttgcgccgccagcggcttcaccttcagcgacagctggatccactgggtgcgccaggcccccggcaagggcctggagtgggtggcctggatcagcccctacggcggcagcacctactacgccgacagcgtgaagggccgcttcaccatcagcgccgacaccagcaagaacaccgcctacctgcagatgaacagcctgcgcgccgaggacaccgccgtgtactactgcgcccgccgccactggcccggcggcttcgactactggggccagggcaccctggtgaccgtgagcagcggaggcgggggaagcggcggcggagggtctggaggagggggcagtgacatccagatgacccagagccccagcagcctgagcgccagcgtgggcgaccgcgtgaccatcacctgccgcgccagccaggacgtgagcaccgccgtggcctggtaccagcagaagcccggcaaggcccccaagctgctgatctacagcgccagcttcctgtacagcggcgtgcccagccgcttcagcggcagcggcagcggcaccgacttcaccctgaccatcagcagcctgcagcccgaggacttcgccacctactactgccagcagtacctgtaccaccccgccaccttcggccagggcaccaaggtggagatcaagggcggtagtggaagaggtgcagctcctgctgcagcacccgcaaaacaagaagcagcggctcccgctcccgccgcaaaagcagaagccccagctgccgcgcccgctgctaaggctggaggttccggacaggtgcagctgcaggagagcggccccggcctggtgaagcccagcgagaccctgagcctgacctgcaccgtgagcggcggcagcgtgagcagcggcgactactactggacctggatccgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 24 - protein, 730 aa, Artificial Sequence
  Synthesized
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGSGRGAAPAAAPAKQEAAAPAPAAKAEAPAAAPAAKAGGSGQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - DNA, 1425 bp, Artificial Sequence
  Synthesized
gaggtgcagctggtggagagcggaggtggactagtacagcctggtggcagcctacgactgagttgcgccgccagcggcttcaccttcagcgacagctggatacactgggtgcgccaggcccccggcaagggcctggagtgggtggcctggatcagcccctacggcggcagcacctactacgccgacagcgtgaagggccgcttcaccatcagcgccgacaccagcaagaacaccgcctacctgcagatgaacagcctgcgcgccgaggacaccgccgtgtactactgcgcccgccgccactggcccggcggcttcgactactggggccagggcaccctggtgaccgtgagcagcggaggcgggggaagcggcggcggagggtctggaggagggggcagtgacatccagatgacccagagccccagcagcctgagcgccagcgtgggcgaccgcgtgaccatcacctgccgcgccagccaggacgtgagcaccgccgtggcctggtaccagcagaagcccggcaaggcccccaagctgctgatctacagcgccagcttcctgtacagcggcgtgcccagccgcttcagcggcagcggcagcggcaccgacttcaccctgaccatcagcagcctgcagcccgaggacttcgccacctactactgccagcagtacctgtaccaccccgccaccttcggccagggcaccaaggtggagatcaaggggggtggcgaagcagctgctaaggaggcagccgcaaaggaagcagctgcaaaggcaggaggcgacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaagcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgt

SEQ ID NO: 26 - protein, 475 aa, Artificial Sequence
  Synthesized
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGGEAAAKEAAAKEAAAKAGGDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - DNA, 1488 bp, Artificial Sequence
  Synthesized
gaggtgcagctggtggagagcggaggtggactagtacagcctggtggcagcctacgactgagttgcgccgccagcggcttcaccttcagcgacagctggatccactgggtgcgccaggcccccggcaagggcctggagtgggtggcctggatcagcccctacggcggcagcacctactacgccgacagcgtgaagggccgcttcaccatcagcgccgacaccagcaagaacaccgcctacctgcagatgaacagcctgcgcgccgaggacaccgccgtgtactactgcgcccgccgccactggcccggcggcttcgactactggggccagggcaccctggtgaccgtgagcagcggaggcgggggaagcggcggcggagggtctggaggagggggcagtgacatccagatgacccagagccccagcagcctgagcgccagcgtgggcgaccgcgtgaccatcacctgccgcgccagccaggacgtgagcaccgccgtggcctggtaccagcagaagcccggcaaggcccccaagctgctgatctacagcgccagcttcctgtacagcggcgtgcccagccgcttcagcggcagcggcagcggcaccgacttcaccctgaccatcagcagcctgcagcccgaggacttcgccacctactactgccagcagtacctgtaccaccccgccaccttcggccagggcaccaaggtggagatcaagggcggtagtggaagaggtgcagctcctgctgcagcacccgcaaaacaagaagcagcggctcccgctcccgccgcaaaagcagaagccccagctgccgcgcccgctgctaaggctggaggttccggagacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaagcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgt

SEQ ID NO: 28 - protein, 496 aa, Artificial Sequence
  Synthesized
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGSGRGAAPAAAPAKQEAAAPAPAAKAEAPAAAPAAKAGGSGDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - DNA, 63 bp, Artificial Sequence
  Synthesized
gggggtggcgaagcagctgctaaggaggcagccgcaaaggaagcagctgcaaaggcaggaggc

SEQ ID NO: 30 - protein, 21 aa, Artificial Sequence
  Synthesized
GGGEAAAKEAAAKEAAAKAGG

SEQ ID NO: 31 - DNA, 126 bp, Artificial Sequence
  Synthesized
ggcggtagtggaagaggtgcagctcctgctgcagcacccgcaaaacaagaagcagcggctcccgctcccgccgcaaaagcagaagccccagctgccgcgcccgctgctaaggctggaggttccgga

SEQ ID NO: 32 - protein, 42 aa, Artificial Sequence
  Synthesized
GGSGRGAAPAAAPAKQEAAAPAPAAKAEAPAAAPAAKAGGSG

SEQ ID NO: 33 - protein, 7 aa, Artificial Sequence
  Synthesized
SGDYYWT

SEQ ID NO: 34 - protein, 16 aa, Artificial Sequence
  Synthesized
HIYYSGNTNYNPSLKS

SEQ ID NO: 35 - protein, 8 aa, Artificial Sequence
  Synthesized
DRVTGAFD

SEQ ID NO: 36 - protein, 11 aa, Artificial Sequence
  Synthesized
QASQDISNYLN

SEQ ID NO: 37 - protein, 7 aa, Artificial Sequence
  Synthesized
DASNLET

SEQ ID NO: 38 - protein, 9 aa, Artificial Sequence
  Synthesized
QHFDHLPLA

SEQ ID NO: 39 - DNA, 1335 bp, Artificial Sequence
  Synthesized
gaggtgcagctggtggagagcggaggcggtctggtgcagcccggaggtagcctacgtctgagctgcgctgcaagcggctactcaatcaccaacgactacgcctggaactgggtgcgccaggcacctggcaagggcctggagtgggtgggctacatcagctacagcggctacaccacctacaaccccagcctgaagagccgcttcaccatcagccgcgacaccagcaagaacaccctgtacctgcagatgaacagcctgcgcgccgaggacaccgccgtgtactactgcgctcgctggactagcggtctggactactggggacagggtactctggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 40 - protein, 445 aa, Artificial Sequence
  Synthesized
EVQLVESGGGLVQPGGSLRLSCAASGYSITNDYAWNWVRQAPGKGLEWVGYISYSGYTTYNPSLKSRFTISRDTSKNTLYLQMNSLRAEDTAVYYCARWTSGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - DNA, 642 bp, Artificial Sequence
  Synthesized
gacatccagatgacccagagccccagcagcctgagcgccagcgtgggcgaccgcgtgaccatcacctgcaaggccagcgacctgatccacaactggctggcctggtaccagcagaagcccggcaaggcccccaagctgctgatctacggcgccaccagcctggagaccggcgtgcccagccgcttcagcggcagcggcagcggcaccgacttcaccctgaccatcagcagcctgcagcccgaggacttcgccacctactactgccagcagtactggaccacccccttcaccttcggccagggcaccaaggtggagatcaagcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgt

SEQ ID NO: 42 - protein, 214 aa, Artificial Sequence
  Synthesized
DIQMTQSPSSLSASVGDRVTITCKASDLIHNWLAWYQQKPGKAPKLLIYGATSLETGVPSRFSGSGSGTDFTLTISSLQPEDFATYYCQQYWTTPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - DNA, 1134 bp, Artificial Sequence
  Synthesized
ctggacagcccagataggccgtggaacccacctactatctctcctgcactgctggtggttacagaaggagataatgctacctttacttgctccttttccaacactagtgagagttttcacgtggtgtggcacagagagtctccttcaggccagacggacaccctcgctgcatttcctgaggaccgcagtcagccggggcaagatagcagattccgcgtgacccagctccccaacggacgcgattttcacatgtccgttgtcagggcacgacgcaacgatagtgggacttatgtgtgcggggtgatcagtctggccccgaagatccagataaaagagtccctccgcgctgaactcagggtgaccgagagacgggccgaagtgcccacagcacacccaagtccaagcccaagacctgctgggcaattccaagggggtggcgaagcagctgctaaggaggcagccgcaaaggaagcagctgcaaaggcaggaggcgacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaagcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgt

SEQ ID NO: 44 - protein, 378 aa, Artificial Sequence
  Synthesized
LDSPDRPWNPPTISPALLVVTEGDNATFTCSFSNTSESFHVVWHRESPSGQTDTLAAFPEDRSQPGQDSRFRVTQLPNGRDFHMSVVRARRNDSGTYVCGVISLAPKIQIKESLRAELRVTERRAEVPTAHPSPSPRPAGQFQGGGEAAAKEAAAKEAAAKAGGDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - DNA, 1134 bp, Artificial Sequence
  Synthesized
ctggacagcccagataggccatggaacccacctactttctctcctgcactgctggtggttacagaaggagataatgctacctttacttgctccttttccaacactagtgagagttttgtccttaattggtatagaatgtctccttcaaatcagacggacaagctcgctgcatttcctgaggaccgcagtcagccggggcaagattgcagattccgcgtgacccagctccccaacggacgcgattttcacatgtccgttgtcagggcacgacgcaacgatagtgggacttatctgtgcggggcgatcagtctggccccgaaggcccagataaaagagtccctccgcgctgaactcagggtgaccgagagacgggccgaagtgcccacagcacacccaagtccaagcccaagacctgctgggcaattccaagggggtggcgaagcagctgctaaggaggcagccgcaaaggaagcagctgcaaaggcaggaggcgacatccagatgacccagagccccagcagcctgagcgccagcgtgggcgaccgcgtgaccatcacctgcaaggccagcgacctgatccacaactggctggcctggtaccagcagaagcccggcaaggcccccaagctgctgatctacggcgccaccagcctggagaccggcgtgcccagccgcttcagcggcagcggcagcggcaccgacttcaccctgaccatcagcagcctgcagcccgaggacttcgccacctactactgccagcagtactggaccacccccttcaccttcggccagggcaccaaggtggagatcaagcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgt

SEQ ID NO: 46 - protein, 378 aa, Artificial Sequence
  Synthesized
LDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQGGGEAAAKEAAAKEAAAKAGGDIQMTQSPSSLSASVGDRVTITCKASDLIHNWLAWYQQKPGKAPKLLIYGATSLETGVPSRFSGSGSGTDFTLTISSLQPEDFATYYCQQYWTTPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - DNA, 1134 bp, Artificial Sequence
  Synthesized
ctggacagcccagataggccatggaacccacctactttctctcctgcactgctggtggttacagaaggagataatgctacctttacttgctccttttccaacactagtgagagttttcacgtggtgtggcacagagagtctccttcaggccagacggacaccctcgctgcatttcctgaggaccgcagtcagccggggcaagattgcagattccgcgtgacccagctccccaacggacgcgattttcacatgtccgttgtcagggcacgacgcaacgatagtgggacttatgtgtgcggggtgatcagtctggccccgaagatccagataaaagagtccctccgcgctgaactcagggtgaccgagagacgggccgaagtgcccacagcacacccaagtccaagcccaagacctgctgggcaattccaagggggtggcgaagcagctgctaaggaggcagccgcaaaggaagcagctgcaaaggcaggaggcgacatccagatgacccagagccccagcagcctgagcgccagcgtgggcgaccgcgtgaccatcacctgcaaggccagcgacctgatccacaactggctggcctggtaccagcagaagcccggcaaggcccccaagctgctgatctacggcgccaccagcctggagaccggcgtgcccagccgcttcagcggcagcggcagcggcaccgacttcaccctgaccatcagcagcctgcagcccgaggacttcgccacctactactgccagcagtactggaccacccccttcaccttcggccagggcaccaaggtggagatcaagcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgt

SEQ ID NO: 48 - protein, 378 aa, Artificial Sequence
  Synthesized
LDSPDRPWNPPTFSPALLVVTEGDNATFTCSFSNTSESFHVVWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQGGGEAAAKEAAAKEAAAKAGGDIQMTQSPSSLSASVGDRVTITCKASDLIHNWLAWYQQKPGKAPKLLIYGATSLETGVPSRFSGSGSGTDFTLTISSLQPEDFATYYCQQYWTTPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 10 aa, Artificial Sequence
  Synthesized
GYSITNDYAW

SEQ ID NO: 50 - protein, 16 aa, Artificial Sequence
  Synthesized
WVGYISYSGYTTYNPS

SEQ ID NO: 51 - protein, 9 aa, Artificial Sequence
  Synthesized
ARWTSGLDY

SEQ ID NO: 52 - protein, 13 aa, Artificial Sequence
  Synthesized
KASDLIHNWLAWY

SEQ ID NO: 53 - protein, 11 aa, Artificial Sequence
  Synthesized
LLIYGATSLET

SEQ ID NO: 54 - protein, 9 aa, Artificial Sequence
  Synthesized
QQYWTTPFT

SEQ ID NO: 55 - DNA, 1119 bp, Artificial Sequence
  Synthesized
ctggacagcccagataggccatggaacccacctactttctctcctgcactgctggtggttacagaaggagataatgctacctttacttgctccttttccaacactagtgagagttttgtccttaattggtatagaatgtctccttcaaatcagacggacaagctcgctgcatttcctgaggaccgcagtcagccggggcaagattgcagattccgcgtgacccagctccccaacggacgcgattttcacatgtccgttgtcagggcacgacgcaacgatagtgggacttatctgtgcggggcgatcagtctggccccgaaggcccagataaaagagtccctccgcgctgaactcagggtgaccgagagacgggccgaagtgcccacagcacacccaagtccaagcccaagacctgctgggcaattccaaggtggaggtggatcaggtggcggcggcagtggcgggggcgggagcggggacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaagcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgt

SEQ ID NO: 56 - protein, 373 aa, Artificial Sequence
  Synthesized
LDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQGGGGSGGGGSGGGGSGDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - DNA, 642 bp, Artificial Sequence
  Synthesized
gacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaaggtggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 58 - protein, 214 aa, Artificial Sequence
  Synthesized
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - DNA, 1347 bp, Artificial Sequence
  Synthesized
gaggtgcagctggtggagtctggaggaggcttggtccagcctggggggtccctgagactctcctgtgcagcctctgggttcaatattaaggacacttacatccactgggtccgccaggctccagggaaggggctggagtgggtcgcacgtatttatcctaccaatggttacacacgctacgcagactccgtgaagggccgattcaccatctccgcagacacttccaagaacacggcgtatcttcaaatgaacagcctgagagccgaggacacggccgtgtattactgttcgagatggggcggtgacggcttctatgccatggactactggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 60 - protein, 449 aa, Artificial Sequence
  Synthesized
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - DNA, 1119 bp, Artificial Sequence
  Synthesized
ctggacagcccagataggccatggaacccacctactttctctcctgcactgctggtggttacagaaggagataatgctacctttacttgctccttttccaacactagtgagagttttcacgtggtgtggcacagagagtctccttcaggccagacggacaccctcgctgcatttcctgaggaccgcagtcagccggggcaagattgcagattccgcgtgacccagctccccaacggacgcgattttcacatgtccgttgtcagggcacgacgcaacgatagtgggacttatgtgtgcggggtgatcagtctggccccgaagatccagataaaagagtccctccgcgctgaactcagggtgaccgagagacgggccgaagtgcccacagcacacccaagtccaagcccaagacctgctgggcaattccaaggtggaggtggatcaggtggcggcggcagtggcgggggcgggagcggggacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaagcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgt

SEQ ID NO: 62 - protein, 373 aa, Artificial Sequence
  Synthesized
LDSPDRPWNPPTFSPALLVVTEGDNATFTCSFSNTSESFHVVWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQGGGGSGGGGSGGGGSGDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - DNA, 1410 bp, Artificial Sequence
  Synthesized
gaggtgcagctggtggagagcggaggtggactagtacagcctggtggcagcctacgactgagttgcgccgccagcggcttcaccttcagcgacagctggatacactgggtgcgccaggcccccggcaagggcctggagtgggtggcctggatcagcccctacggcggcagcacctactacgccgacagcgtgaagggccgcttcaccatcagcgccgacaccagcaagaacaccgcctacctgcagatgaacagcctgcgcgccgaggacaccgccgtgtactactgcgcccgccgccactggcccggcggcttcgactactggggccagggcaccctggtgaccgtgagcagcggaggcgggggaagcggcggcggagggtctggaggagggggcagtgacatccagatgacccagagccccagcagcctgagcgccagcgtgggcgaccgcgtgaccatcacctgccgcgccagccaggacgtgagcaccgccgtggcctggtaccagcagaagcccggcaaggcccccaagctgctgatctacagcgccagcttcctgtacagcggcgtgcccagccgcttcagcggcagcggcagcggcaccgacttcaccctgaccatcagcagcctgcagcccgaggacttcgccacctactactgccagcagtacctgtaccaccccgccaccttcggccagggcaccaaggtggagatcaagggtggaggtggatcaggtggcggcggcagtggcgggggcgggagcggggacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaagcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgt

SEQ ID NO: 64 - protein, 470 aa, Artificial Sequence
  Synthesized
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGGGSGGGGSGGGGSGDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - DNA, 1353 bp, Artificial Sequence
  Synthesized
tttaccgttaccgtcccgaaggatctctacgtcgtcgaatacggtagtaacatgaccatagagtgtaaattccctgtggagaagcaacttgacctggcagccctgattgtgtattgggagatggaggacaaaaacattatacagttcgtgcacggtgaggaggacctgaaagtgcaacattcctcctatcgccagagagcccgcctgctgaaggaccagctgtcacttgggaacgccgccctccagattaccgacgtaaaacttcaggatgccggtgtgtacaggtgcatgatatcttacgggggtgctgattacaagagaatcactgttaaggtcaatgcgccctacaacaagataaatcagcggattctggtcgttgatccagttacatccgagcacgagctgacctgtcaagctgagggctacccgaaggctgaagtaatctggacatcctccgaccaccaggtcctctctggaaagacaactacaacaaacagcaagcgagaggagaagctgtttaacgtcacgagtacactccgaatcaatacaacaaccaatgaaattttctactgcacttttaggcgcctcgaccctgaggaaaaccatacagccgaactcgtaattcccgagctgcccctcgcccaccctccaaacgaacgcacaggtggaggtggatcaggtggcggcggcagtggcgggggcgggagcggggacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaagcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgt

SEQ ID NO: 66 - protein, 451 aa, Artificial Sequence
  Synthesized
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTGGGGSGGGGSGGGGSGDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - DNA, 1119 bp, Artificial Sequence
  Synthesized
ctggacagcccagataggccgtggaacccacctactatctctcctgcactgctggtggttacagaaggagataatgctacctttacttgctccttttccaacactagtgagagttttcacgtggtgtggcacagagagtctccttcaggccagacggacaccctcgctgcatttcctgaggaccgcagtcagccggggcaagatagcagattccgcgtgacccagctccccaacggacgcgattttcacatgtccgttgtcagggcacgacgcaacgatagtgggacttatgtgtgcggggtgatcagtctggccccgaagatccagataaaagagtccctccgcgctgaactcagggtgaccgagagacgggccgaagtgcccacagcacacccaagtccaagcccaagacctgctgggcaattccaaggtggaggtggatcaggtggcggcggcagtggcgggggcgggagcggggacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaagcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgt

SEQ ID NO: 68 - protein, 373 aa, Artificial Sequence
  Synthesized
LDSPDRPWNPPTISPALLVVTEGDNATFTCSFSNTSESFHVVWHRESPSGQTDTLAAFPEDRSQPGQDSRFRVTQLPNGRDFHMSVVRARRNDSGTYVCGVISLAPKIQIKESLRAELRVTERRAEVPTAHPSPSPRPAGQFQGGGGSGGGGSGGGGSGDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - DNA, 1119 bp, Artificial Sequence
  Synthesized
ctggacagcccagataggccatggaacccacctactttctctcctgcactgctggtggttacagaaggagataatgctacctttacttgctccttttccaacactagtgagagttttgtccttaattggtatagaatgtctccttcaaatcagacggacaagctcgctgcatttcctgaggaccgcagtcagccggggcaagattgcagattccgcgtgacccagctccccaacggacgcgattttcacatgtccgttgtcagggcacgacgcaacgatagtgggacttatctgtgcggggcgatcagtctggccccgaaggcccagataaaagagtccctccgcgctgaactcagggtgaccgagagacgggccgaagtgcccacagcacacccaagtccaagcccaagacctgctgggcaattccaaggtggaggtggatcaggtggcggcggcagtggcgggggcgggagcggggacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaaggtggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 70 - protein, 373 aa, Artificial Sequence
  Synthesized
LDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQGGGGSGGGGSGGGGSGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - DNA, 1119 bp, Artificial Sequence
  Synthesized
ctggacagcccagataggccatggaacccacctactttctctcctgcactgctggtggttacagaaggagataatgctacctttacttgctccttttccaacactagtgagagttttcacgtggtgtggcacagagagtctccttcaggccagacggacaccctcgctgcatttcctgaggaccgcagtcagccggggcaagattgcagattccgcgtgacccagctccccaacggacgcgattttcacatgtccgttgtcagggcacgacgcaacgatagtgggacttatgtgtgcggggtgatcagtctggccccgaagatccagataaaagagtccctccgcgctgaactcagggtgaccgagagacgggccgaagtgcccacagcacacccaagtccaagcccaagacctgctgggcaattccaaggtggaggtggatcaggtggcggcggcagtggcgggggcgggagcggggacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaaggtggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 72 - protein, 373 aa, Artificial Sequence
  Synthesized
LDSPDRPWNPPTFSPALLVVTEGDNATFTCSFSNTSESFHVVWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQGGGGSGGGGSGGGGSGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - DNA, 1410 bp, Artificial Sequence
  Synthesized
gaggtgcagctggtggagagcggaggtggactagtacagcctggtggcagcctacgactgagttgcgccgccagcggcttcaccttcagcgacagctggatacactgggtgcgccaggcccccggcaagggcctggagtgggtggcctggatcagcccctacggcggcagcacctactacgccgacagcgtgaagggccgcttcaccatcagcgccgacaccagcaagaacaccgcctacctgcagatgaacagcctgcgcgccgaggacaccgccgtgtactactgcgcccgccgccactggcccggcggcttcgactactggggccagggcaccctggtgaccgtgagcagcggaggcgggggaagcggcggcggagggtctggaggagggggcagtgacatccagatgacccagagccccagcagcctgagcgccagcgtgggcgaccgcgtgaccatcacctgccgcgccagccaggacgtgagcaccgccgtggcctggtaccagcagaagcccggcaaggcccccaagctgctgatctacagcgccagcttcctgtacagcggcgtgcccagccgcttcagcggcagcggcagcggcaccgacttcaccctgaccatcagcagcctgcagcccgaggacttcgccacctactactgccagcagtacctgtaccaccccgccaccttcggccagggcaccaaggtggagatcaagggtggaggtggatcaggtggcggcggcagtggcgggggcgggagcggggacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaaggtggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 74 - protein, 470 aa, Artificial Sequence
  Synthesized
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGGGSGGGGSGGGGSGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - DNA, 1353 bp, Artificial Sequence
  Synthesized
ttcaccgtgaccgtgcccaaggacctgtacgtggtggagtacggcagcaacatgaccatcgagtgcaagttccccgtggagaagcagctggacctggccgccctgatcgtgtactgggagatggaggacaagaacatcatccagttcgtgcacggcgaggaggacctgaaggtgcagcacagcagctaccgccagcgcgcccgcctgctgaaggaccagctgagcctgggcaacgccgccctgcagatcaccgacgtgaagctgcaggacgccggcgtgtaccgctgcatgatcagctacggcggcgccgactacaagcgcatcaccgtgaaggtgaacgccccctacaacaagatcaaccagcgcatcctggtggtggaccccgtgaccagcgagcacgagctgacctgccaggccgagggctaccccaaggccgaggtgatctggaccagcagcgaccaccaggtgctgagcggcaagaccaccaccaccaacagcaagcgcgaggagaagctgttcaacgtgaccagcaccctgcgcatcaacaccaccaccaacgagatcttctactgcaccttccgccgcctggaccccgaggagaaccacaccgccgagctggtgatccccgagctgcccctggcccacccccccaacgagcgcaccggtggaggtggatcaggtggcggcggcagtggcgggggcgggagcggggacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaaggtggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 76 - protein, 451 aa, Artificial Sequence
  Synthesized
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTGGGGSGGGGSGGGGSGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - DNA, 1119 bp, Artificial Sequence
  Synthesized
ctggacagcccagataggccgtggaacccacctactatctctcctgcactgctggtggttacagaaggagataatgctacctttacttgctccttttccaacactagtgagagttttcacgtggtgtggcacagagagtctccttcaggccagacggacaccctcgctgcatttcctgaggaccgcagtcagccggggcaagatagcagattccgcgtgacccagctccccaacggacgcgattttcacatgtccgttgtcagggcacgacgcaacgatagtgggacttatgtgtgcggggtgatcagtctggccccgaagatccagataaaagagtccctccgcgctgaactcagggtgaccgagagacgggccgaagtgcccacagcacacccaagtccaagcccaagacctgctgggcaattccaaggtggaggtggatcaggtggcggcggcagtggcgggggcgggagcggggacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaaggtggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 78 - protein, 373 aa, Artificial Sequence
  Synthesized
LDSPDRPWNPPTISPALLVVTEGDNATFTCSFSNTSESFHVVWHRESPSGQTDTLAAFPEDRSQPGQDSRFRVTQLPNGRDFHMSVVRARRNDSGTYVCGVISLAPKIQIKESLRAELRVTERRAEVPTAHPSPSPRPAGQFQGGGGSGGGGSGGGGSGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 5 aa, Artificial Sequence
  Synthesized
DTYIH

SEQ ID NO: 80 - protein, 17 aa, Artificial Sequence
  Synthesized
RIYPTNGYTRYADSVKG

SEQ ID NO: 81 - protein, 11 aa, Artificial Sequence
  Synthesized
WGGDGFYAMDY

SEQ ID NO: 82 - protein, 11 aa, Artificial Sequence
  Synthesized
RASQDVNTAVA

SEQ ID NO: 83 - protein, 7 aa, Artificial Sequence
  Synthesized
SASFLYS

SEQ ID NO: 84 - protein, 9 aa, Artificial Sequence
  Synthesized
QQHYTTPPT

SEQ ID NO: 85 - protein, 16 aa, Artificial Sequence
  Synthesized
GGGGSGGGGSGGGGSG

(19) United States
(12) Patent Application Publication
(10) Pub. No.: US 2021/0403575 A1
(43) Pub. Date: Dec. 30, 2021

(54) Bispecific antibody

(71) Applicant: SHANGHAI YICHEN BIOMED CO. LTD, Shanghai (CN)

(72) Inventors: Feng WANG, Shanghai (CN); Huayang ZHENG, Shanghai (CN); Yuhan ZHANG, Shanghai (CN)

(21) Appl. No.: 17/287,539

(22) PCT Filed: Oct. 22, 2019
(86) PCT No.: PCT/CN2019/112509

§ 371 Date: Apr. 22, 2021

(30) Foreign Application Priority Data
Oct. 22, 2018 (CN) 201811230752.1

(51) Int. Cl. C07K 16/28; C07K 16/32

(52) U.S. Cl. CPC C07K 16/2863; C07K 16/2827; C07K 2317/565; C07K 2317/31; C07K 16/32

(57) ABSTRACT

Disclosed is a bispecific antibody, and in particular, a bispecific antibody that simultaneously targets a tumor cell surface antigen and an immune checkpoint protein. A first binding domain of the above molecule is an antibody containing a constant region, a heavy chain variable region, and a light chain variable region. Through the high affinity binding between the first binding domain and the tumor cell surface antigen, the second binding domain for an immune checkpoint fused with the first binding domain is enriched on or near a tumor cell or in a tumor microenvironment, so as to exert a specific killing effect of an effector cell on the tumor cell.

### TECHNICAL FIELD

The present disclosure relates to a bispecific antibody, and in particular to a bispecific antibody that simultaneously targets a tumor cell surface antigen and an immune checkpoint protein, and its pharmaceutical composition and use thereof.

### BACKGROUND

In recent years, as a cytotoxic T lymphocyte-associated antigen-4 (CTLA-4) antibody is approved for marketing by the US Food and Drug Administration (FDA), tumor immunotherapy has attracted more and more attention of people. In a tumor immune response, T-cell-mediated cellular immunity plays a major role. T-cells recognize a tumor antigen through a T-Cell Receptor (TCR) so as to activate themselves and kill tumor cells. The activation of T-cells requires not only a first signal system provided by a tumor antigen, but also second signal systems, which may include co-stimulatory signals and co-inhibitory signals mediating the activation and inhibition of T-cells respectively. Although the tumor cell expresses a variety of tumor antigens, it evades T-cell killing and host immune system attack by expressing some immunosuppressive molecules. These immunosuppressive molecules mainly include PD-1, CTLA-4, TIM3, LAGS, etc. PD-1 and PD-L1/PD-L2 pathways are the most thoroughly investigated immunosuppressive checkpoints in the tumor immunotherapy at present (Biochimica et Biophysica Acta(BBA)—Reviews on Cancer 2017; 1868(2): 571-583).

PD-1, a member of CD28 family, is an immunosuppressive molecule. Its structure thereof includes: an extracellular immunoglobulin variable region (IgV)-like domain, a hydrophobic transmembrane region, and an intracellular region. PD-1 is expressed on CD4⁻CD8⁻thymocyte, and inducibly expressed on activated T-cells, B-cells, bone marrow cells, dendritic cells, natural killer cells, monocytes and the like. Continuous expression of PD-1 on the T-cell may induce T-cell exhaustion. PD-1 expression on tumor-infiltrating lymphocytes may affect T-cell function, weaken cytokine secretion, as well the T-cell tumor-killing effect. It is also closely related to poor prognosis and high tumor recurrence rate of patients with renal cell carcinoma and non-small cell lung cancer (Pan Jiajia, et al.; Journal of China Pharmaceutical University 2016, 47(1): 9-18).

PD-1 has two ligands, namely PD-L1 and PD-L2 ligands, both of which belong to the B7 family. PD-L1 is widely expressed on activated B-cell, T-cell, macrophage, DCs, and NK cells and the like. PD-L1 is also expressed on the surface of many tumor cells, such as lung cancer, breast cancer, malignant melanoma, esophageal cancer, gastric cancer, and pancreatic cancer.

The surface of the tumor cells, through high expression of PD-L1 or PD-L2 molecules, binds to the receptor PD-1 on the T-cell and transmits a negative regulatory signal, to cause immune apoptosis and immune incompetence of a tumor antigen-specific T-cell, so that the tumor cells evade immune surveillance and killing of a body (Pan Jiajia et al.; Journal of China Pharmaceutical University 2016, 47(1): 9-18). Therefore, the PD-1/PD-Ls signal pathway is used as a target to develop a blocking agent against PD-1 or PD-Ls, and it may enhance the killing of the tumor cells by the T-cell.

An epidermal growth factor receptor (EGFR) is a membrane protein product of proto-oncogene C-erbB-1 (HER-1), and is mainly expressed on an epithelial cell membrane, it mainly includes an extracellular region, a transmembrane region and an intracellular region. It is indicated from researches that many malignant tumors that occur in humans have an abnormally high expression phenomenon of an EGFR molecule, and it is also discovered that the EGFR expression is often related to cancer cell proliferation, neoangiogenesis, tumor metastasis, and inhibition of cancer cell apoptosis (anti-apoptosis), possible mechanisms thereof are as follows: EGFR overexpression is activated and downstream signal transduction is enhanced; the downstream signal transduction is also enhanced by a mutant EGFR receptor existing in the body; or continuous activation of EGFR is caused by overexpression of an EGFR ligand; or the continuous activation of EGFR is caused by excessive expression of the EGFR ligand; it is also possible that an autocrine loop function is enhanced; a down-regulation mechanism of EGFR is destroyed; and an abnormal signal transduction pathway is activated, etc. It is indicated from existing documents that EGFR is overexpressed in a variety of human malignant tumors, and plays an important role in the occurrence and development processes of human cancers. These tumors include breast cancer, gastric cancer, lung cancer, head and neck tumors, ovarian cancer, colon cancer, brain cancer, glial cell, bladder cancer, kidney cancer and prostate cancer and the like (Sooro M A, Zhang N, Zhang P. Targeting EGFR-mediated autophagy as a potential strategy for cancer therapy. Int J Cancer. 2018 Mar. 25. doi: 10.1002/ijc.31398. [Epub ahead of print]).

An anti-EGFR monoclonal antibody may specifically bind to EGFR and compete to block its binding to a ligand, thereby transduction of a downstream signal is inhibited. Panitumumab is a fully humanized IgG2 anti-EGFR monoclonal antibody produced by a XenoMouse technology, and is approved by the FDA in September 2006 for treatment of EGFR-positive metastatic colorectal cancer. Its mechanism of action is to competitively bind to EGFR on the tumor cells, block the binding of EGFR to ligands EGF and TGFa, induce EGFR internalization, and eliminate a cellular effect mediated by EGFR.

However, a traditional monoclonal antibody only binds to a single epitope of a single target, so efficacy thereof is limited to a certain extent. It is revealed from pharmacological researches that most complex diseases are related to multiple disease-related signal pathways. For example, a tumor necrosis factor TNF, multiple pro-inflammatory cytokines such as interleukin 6 (IL-6) mediate immune inflammatory diseases simultaneously, while the proliferation of the tumor cells is often caused by abnormal up-regulation of multiple growth factor receptors. Blocking of a single signal pathway usually has limited efficacy, and drug resistance is easily formed. Therefore, the development of a bispecific antibody and an analogue thereof that may bind to two different targets simultaneously is an important field in the development of a new structural antibody for a long time.

The bispecific antibody, through targeting two different antigens, builds a bridge between a target cell and a functional molecule (cell), stimulates an immune response having a guidance property, and has a broad application prospect in immunotherapy of tumors and inflammatory diseases. According to different combination types, the bispecific antibodies may be divided into a cytokine-antibody fusion protein, a double-chain antibody, a single-chain bivalent antibody, and a multivalent bispecific antibody (Li Feng et al.; China Medical Biotechnology 2014, 9(4): 291-293). The cytokine-antibody fusion protein carries a cytokine to a tumor site through a monoclonal antibody targeting an antigen, and a systemic toxic and side effect of a free factor is avoided while the anti-tumor effect is maximized. The cytokine antibody fusion protein containing IL-2, IL-12, IL-21, TNFa and INF-a, beta, and gamma is researched and designed and shows a good anti-tumor effect in preclinical researches and early clinical trials (Patricia A. Young et al. Semin Oncol 2014, 41(5): 623-636). The preparation of a bifunctional antibody is an ongoing demand in tumor therapy.

### SUMMARY

The present disclosure relates to a bispecific antibody, which comprises: a first binding domain which targets a surface antigen of a first target cell, and a second binding domain which binds an immune checkpoint protein on the surface of a second target cell, herein the first binding domain is an antibody structure including a constant region, a heavy chain variable region, and a light chain variable region, the second binding domain is linked to an N-terminal of the heavy chain variable region or the light chain variable region of the first binding domain, herein the first target cell is a tumor cell, the second target cell is the same cell as the first target cell, or the second target cell is an immune cell.

In a specific implementation mode, the bispecific antibody of the present disclosure targets two different antigens on the same tumor cell.

In a specific implementation mode, the bispecific antibody of the present disclosure targets two different antigens on the tumor cell and the immune cell, respectively.

In another aspect, the present disclosure further relates to a nucleic acid, which encodes the bispecific antibody of the present disclosure.

In another aspect, the present disclosure further relates to an expression vector, which includes the nucleic acid of the present disclosure.

In another aspect, the present disclosure further relates to a host cell, which includes the expression vector of the present disclosure.

In another aspect, the present disclosure further relates to a pharmaceutical composition, which includes the bispecific antibody of the present disclosure.

In another aspect, the present disclosure further relates to the use of the bispecific antibody in preparing medication, wherein the medication is used for autoimmune disease and cancer therapy.

The present disclosure discloses the following technical solutions:

1. A bispecific antibody, including: a first binding domain which targets a surface antigen of a first target cell, and a second binding domain which binds to an immune checkpoint protein on the surface of a second target cell, herein the first binding domain is an antibody structure comprising a constant region, a heavy chain variable region, and a light chain variable region, the second binding domain is linked to an N-terminal of the heavy chain variable region or the light chain variable region of the first binding domain, herein the first target cell is a tumor cell, the second target cell is the same cell as the first target cell, or the second target cell is an immune cell.

2. The bispecific antibody according to the technical solution 1, herein the antibody targets two different antigens on the same tumor cell.

3. The bispecific antibody according to the technical solution 1, herein the antibody targets two different antigens on the tumor cell and the immune cell.

4. The bispecific antibody according to any one of the previous technical solutions, herein the immune cell is selected from an NK cell, a T lymphocyte and a B-cell.

5. The bispecific antibody according to any one of the previous technical solutions, herein the tumor cell surface antigen is selected from one of a growth factor receptor, a receptor tyrosine kinase and a mucin family.

6. The bispecific antibody according to the technical solution 5, herein the growth factor receptor is selected from one of an epidermal growth factor receptor family, a tyrosine kinase receptor family, a vascular endothelial growth factor receptor family, an insulin-like growth factor 1 receptor and a platelet-derived growth factor receptor family.

7. The bispecific antibody according to the technical solution 6, herein the growth factor receptor is selected from an epidermal growth factor receptor (EGFR), a vascular endothelial growth factor receptor 1 (VEGFR-1, FLT1), a vascular endothelial growth factor receptor 2 (VEGFR-2, KDR/Flk-1), a vascular endothelial growth factor receptor 3 (VEGFR-3), an insulin-like growth factor 1 receptor (IGF-1R), a platelet-derived growth factor receptor A subunit (PDGF-RA) and a platelet-derived growth factor receptor B subunit (PDGF-RB).

8. The bispecific antibody according to the technical solution 5, herein the receptor tyrosine kinase is selected from one of an ERBB2 receptor tyrosine kinase 2 (HER2), an ERBB2 receptor tyrosine kinase 3 (HER3) and an ERBB2 receptor tyrosine kinase 4 (HER4).

9. The bispecific antibody according to the technical solution 5, herein the mucin family is selected from one of mucin1 (MUC1), MUC2, MUC3A, MUC3B, MUC4, MUC5AC, MUC5B, MUC6, MUC7, MUC8, MUC12, MUC13, MUC15, MUC16, MUC17, MUC19 and MUC20.

10. The bispecific antibody according to any one of the previous technical solutions, herein the immune checkpoint protein is selected from one of PD-1, PD-L1, CTLA-4, LAG-3, OX40, CD28, CD40, CD47, CD70, CD80, CD122, GTIR, A2AR, B7-H3 (CD276), B7-H4, IDO, KIR, Tim-3 and 4-1BB (CD137).

11. The bispecific antibody according to any one of the previous technical solutions, herein the antibody targets EGFR antigen and PD-L1 antigen, or targets to MUC16 antigen and PD-L1 antigen, or targets to EGFR antigen and PD-L1 antigen.

12. The bispecific antibody according to any one of the previous technical solutions, herein the second binding domain is PD1.

13. The bispecific antibody according to any one of the previous technical solutions, herein the second binding domain is human PD1 or a variant thereof.

14. The bispecific antibody according to any one of the previous technical solutions, herein the second binding domain is ScFv of an anti-PD-L1 antibody or a fragment thereof.

15. The bispecific antibody according to any one of the previous technical solutions, herein the first binding domain only has function of binding to a cell surface antigen, or has both Fc effector function and function of binding to a cell surface antigen.

16. The bispecific antibody according to the technical solution 15, herein the second binding domain is selected from amino acids 1-143 of SEQ ID NO: 6, amino acids 1-143 of SEQ ID NO: 14, amino acids 1-143 of SEQ ID NO: 44 or amino acids 1-240 of SEQ ID No: 22.

17. The bispecific antibody according to any one of the previous technical solutions, herein the heavy chain variable region of the first binding domain includes the region selected from the followings: CDR1-H, CDR2-H and CDR3-H, and the light chain variable region includes the region selected from the followings: CDR1-L, CDR2-L and CDR3-L;

a) CDR1-H shown in SEQ ID No: 33, CDR2-H shown in SEQ ID No: 34 and CDR3-H shown in SEQ ID No: 35; CDR1-L shown in SEQ ID No: 36, CDR2-L shown in SEQ ID No: 37, and CDR3-L shown in SEQ ID No: 38;

b) CDR1-H shown in SEQ ID No. 49, CDR2-H shown in SEQ ID No: 50, and CDR3-H shown in SEQ ID No: 51; and CDR1-L shown in SEQ ID No: 52, CDR2-L shown in SEQ ID No: 52, and CDR3-L shown in SEQ ID No: 53; or

c) CDR1-H shown in SEQ ID No. 79, CDR2-H shown in SEQ ID No: 80, and CDR3-H shown in SEQ ID No: 81; and CDR1-L shown in SEQ ID No: 82, CDR2-L shown in SEQ ID No: 83, and CDR3-L shown in SEQ ID No: 84.

18. The bispecific antibody according to any one of the previous technical solutions, herein the second binding domain is linked to an N-terminal of the heavy chain variable region or the light chain variable region of the first binding domain, and is linked by a peptide linker.

19. The bispecific antibody according to the technical solution 18, herein the peptide linker has the amino acids set forth in the L1 of SEQ ID No: 30, L2 of SEQ ID No: 32, or L3 of SEQ ID No: 85.

20. The bispecific antibody according to any one of the previous technical solutions, herein the Fc region of the first binding domain is selected from amino acids 223-448 of SEQ ID No: 2.

21. A nucleic acid encoding the bispecific antibody according to any one of the technical solutions 1-20.

22. An expression vector including the nucleic acid according to the technical solution 21.

23. A host cell, herein it includes the expression vector according to the technical solution 22.

24. A pharmaceutical composition, herein it includes the bispecific antibody according to any one of the technical solutions 1-20.

25. A use of the antibody according to any one of the technical solutions 1-20 in preparing a medication, herein the medication is used for treating autoimmune disease and cancer.

The present disclosure fuses the immune checkpoint antigen, such as PD-1, to the antibody against the tumor cell surface antigen, such as a heavy chain or a light chain of anti-EGFR, or a light chain of anti-HER2, to obtain PD-1-anti-EGFR (or, PD-1-anti-HER2) which may simultaneously target EGFR and PD-L1 (or, HER2 and PD-L1) on the tumor cells, antagonize the function of EGFR (or, HER2), block the binding of PD-L1 on the tumor cells to PD-1 on the T-cell, and specifically promote the immune T cells, which undergoes the transition from anergy to activation, to exert its specific killing effects on surrounding tumor cells.

The present disclosure uses the antibody against the tumor cell surface antigen (for example, anti-EGFR, anti-MUC16 or anti-HER2) as delivery vehicle, and fuses the effector molecules, namely the binding domain targeted to the immune checkpoint protein (for example, a PD-1 protein or a fragment of anti-PD-L1, to antibody heavy chain or light chain, the bispecific antibody which may respectively bind to the tumor cell surface specific antigen (EGFR, MUC16 or HER2) and the immune checkpoint (such as PD-L1) is formed. By high affinity binding to the specific targets on tumor cell surface, the bispecific molecules exerting functions to the immune checkpoints are enriched to the tumor cells or tumor microenvironment, which can localize their modulation functions on immune checkpoint of effector cells within tumor or tumor microenvironment, thereby significantly reduce the systemic immune activation by immune checkpoint modulators. In addition, because of the high affinity of these bispecific molecules on tumor specific targets, their affinities can be adjusted in certain range to optimize the functions on immune checkpoint targets, which has great potential on a variety of clinical indications.

### BRIEF DESCRIPTION OF THE DRAWINGS

FIG. 1: SDS-PAGE electrophoresis diagram of antibody fusion protein

M represents a protein marker.

“−” represents sample without treatment by beta-mercaptoethanol.

“+” represents sample with treatment by the beta-mercaptoethanol.

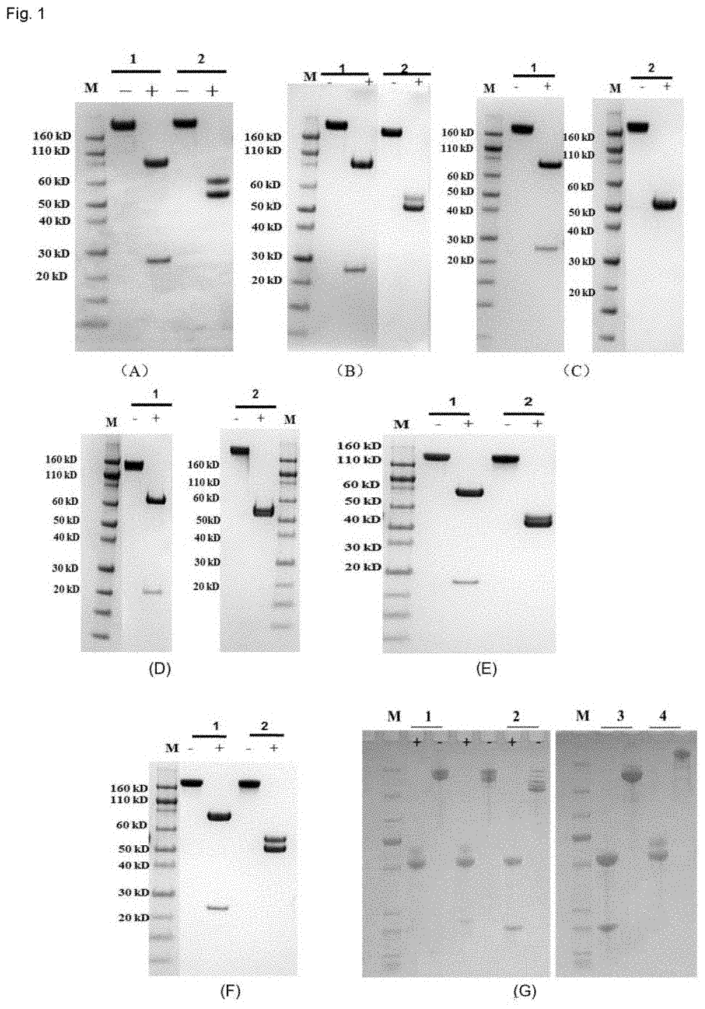

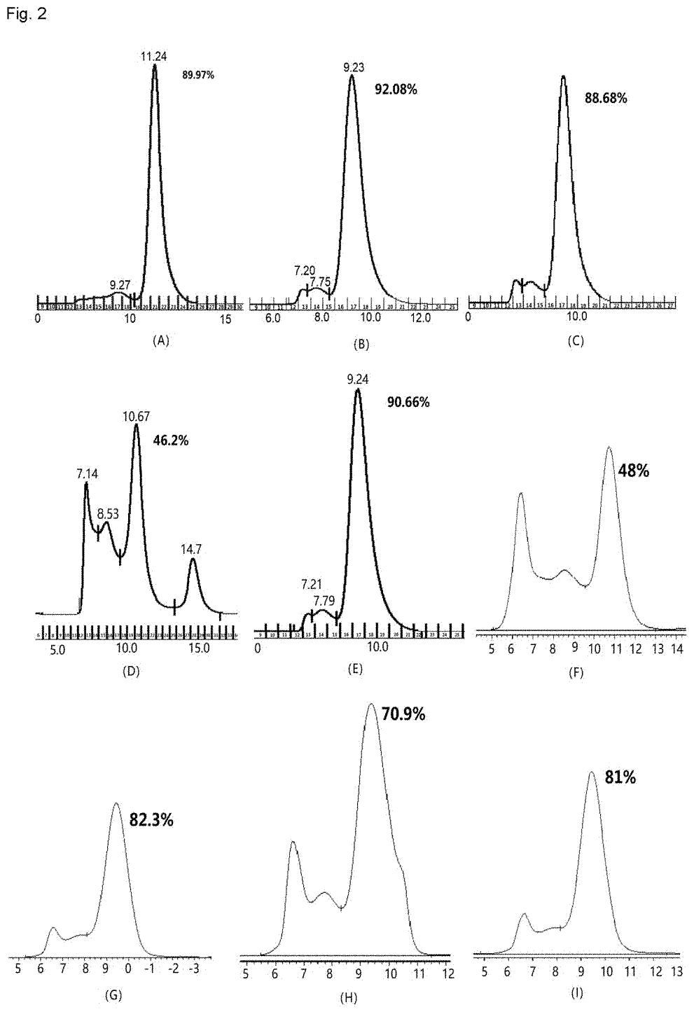

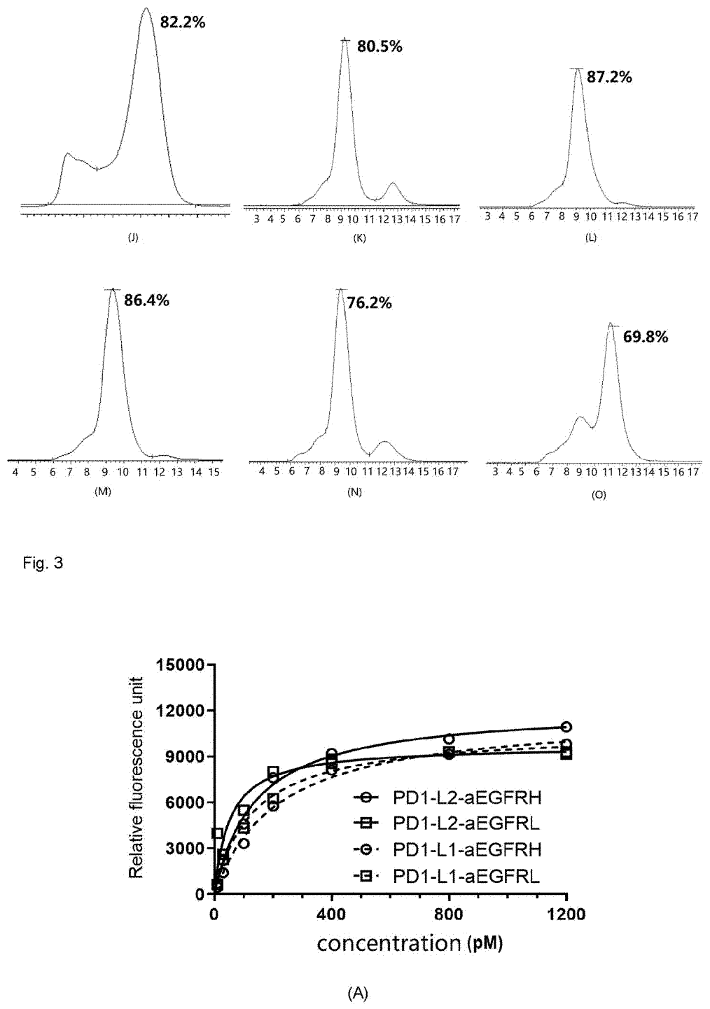

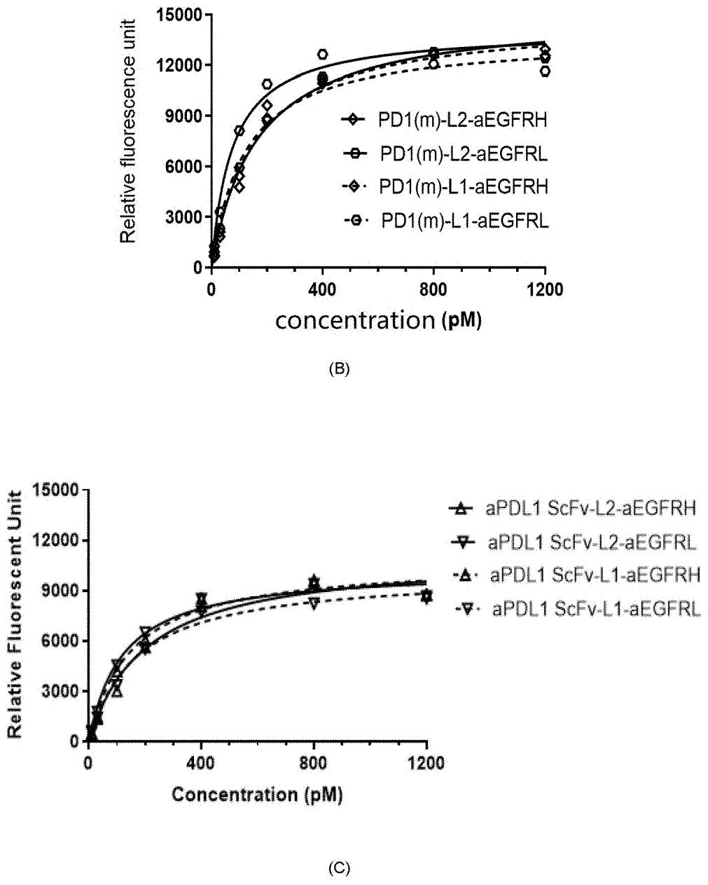

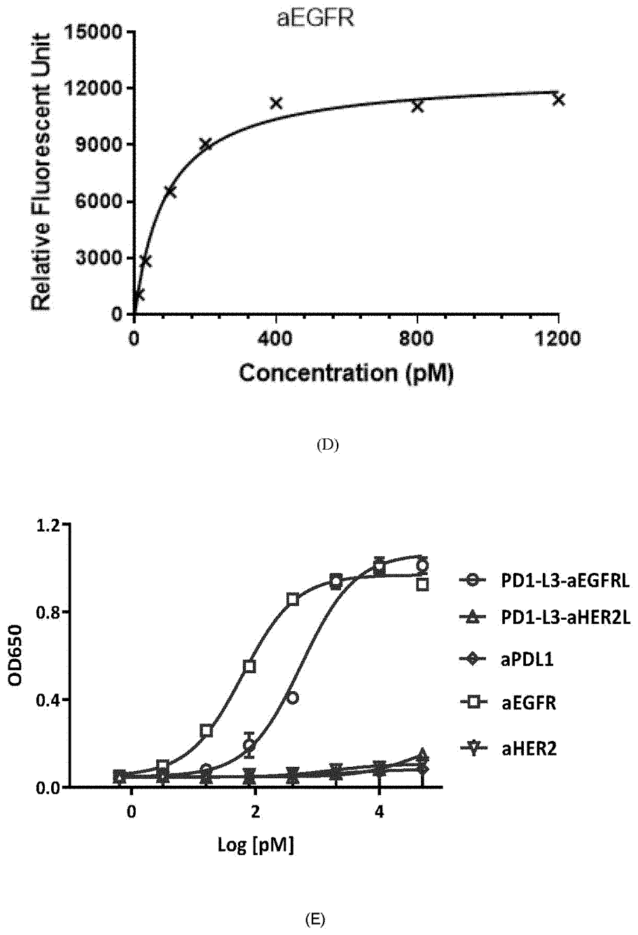

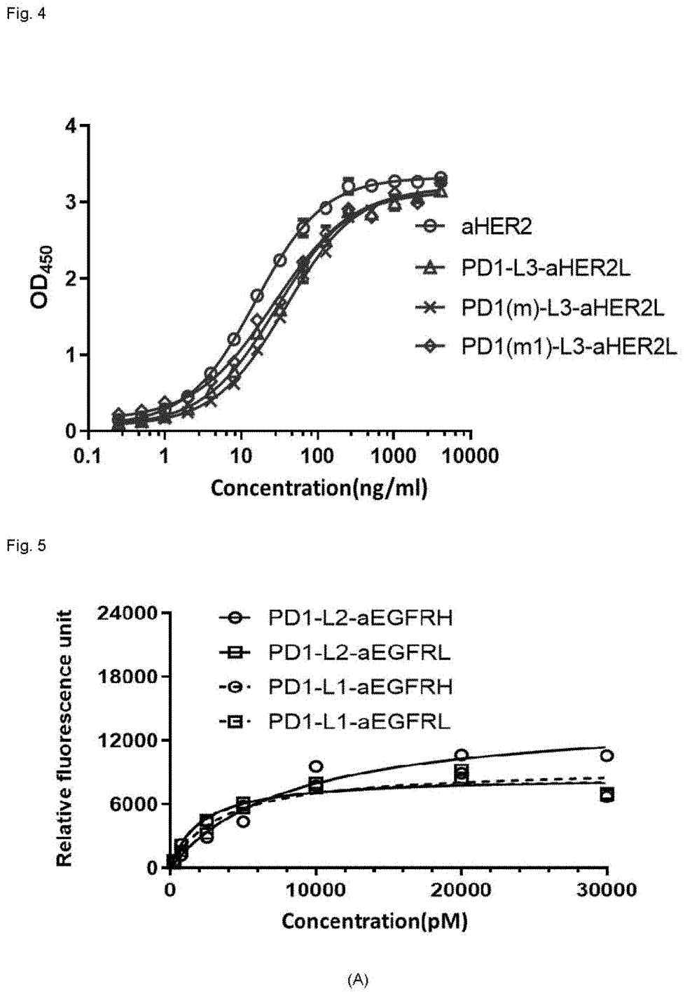

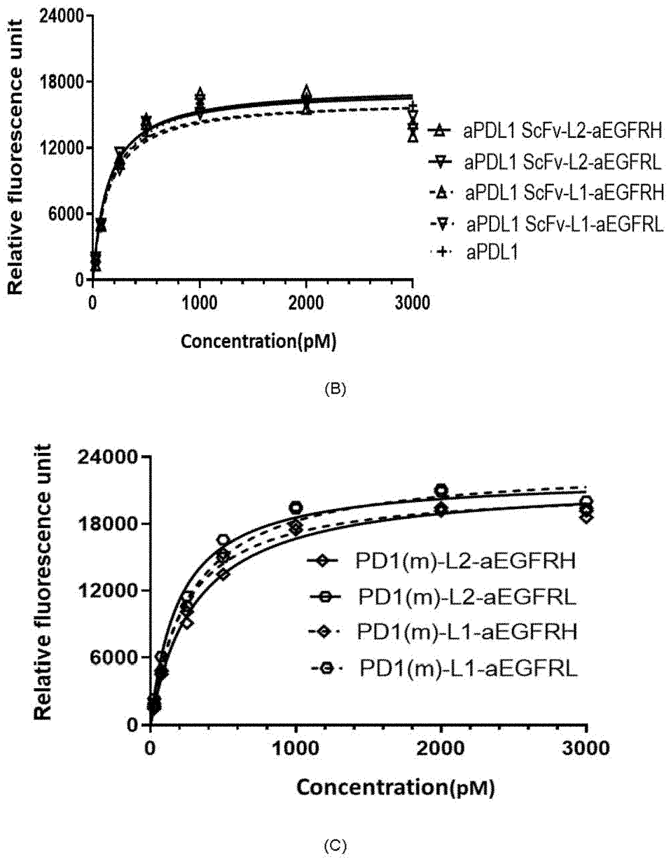

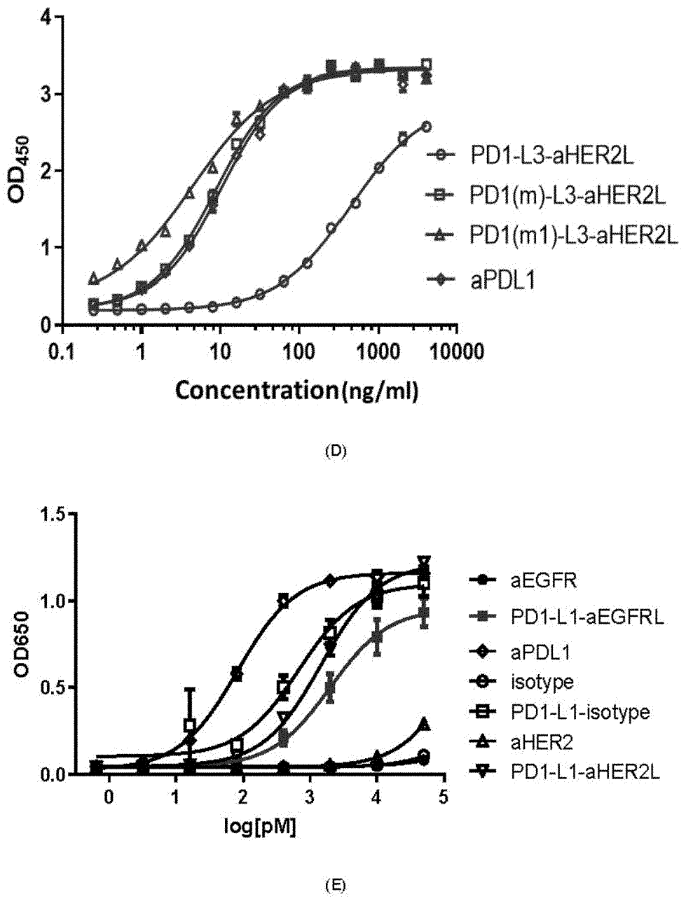

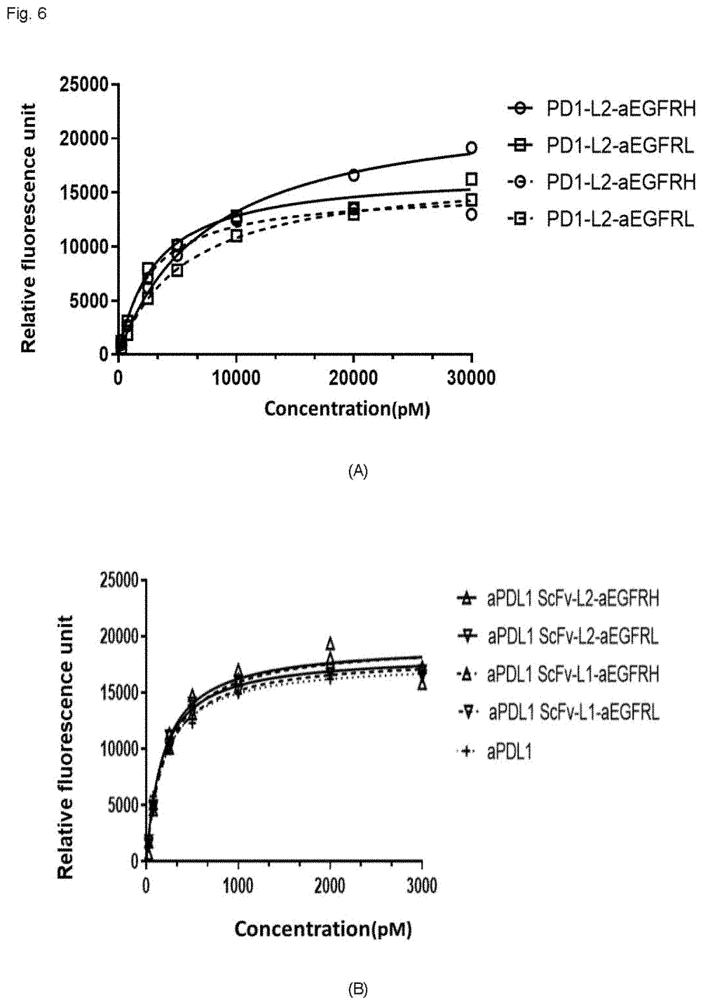

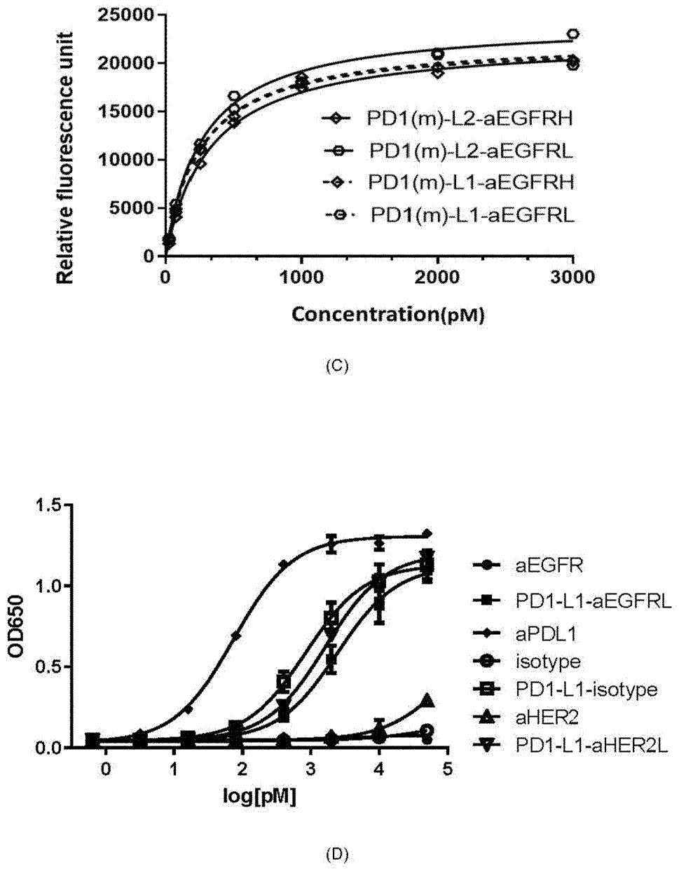

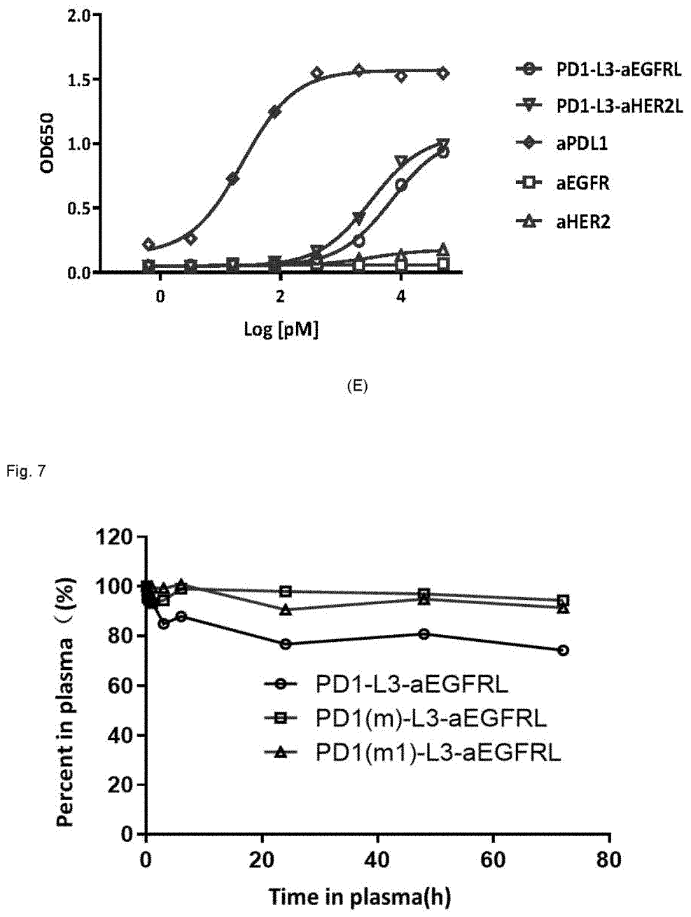

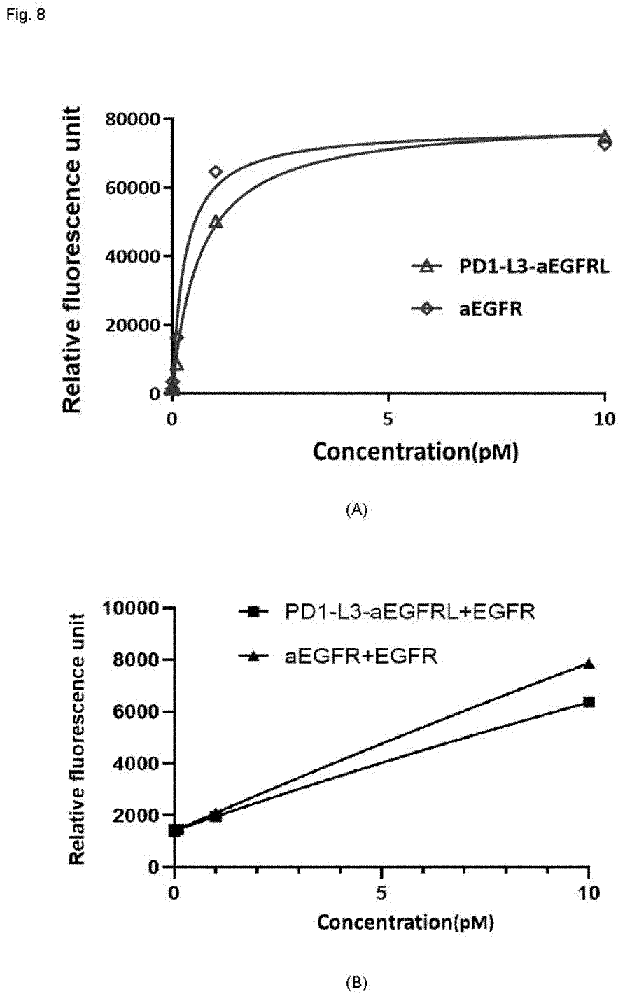

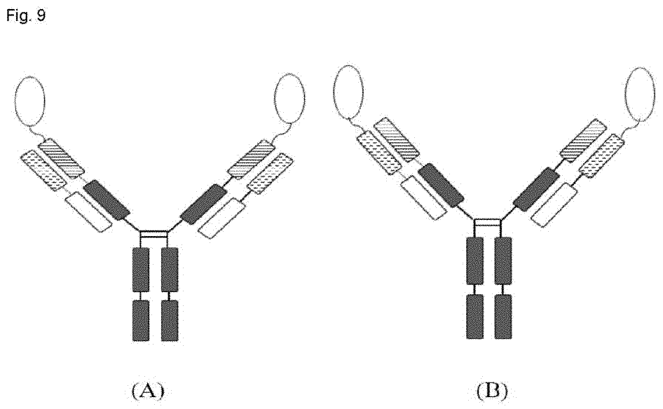

Lane 1 of FIG. 1A is loading of PD1-L1-aEGFRH; Lane 2 is loading of PD1-L1-aEGFRL;

Lane 1 of FIG. 1B is loading of PD1-L2-aEGFRH; Lane 2 is loading of PD1-L2-aEGFRL;

Lane 1 of FIG. 1C is loading of aPDL1ScFv-L1-aEGFRH antibody; Lane 2 is aPDL1ScFv-L1-aEGFRL;

Lane 1 of FIG. 1D is loading of aPDL1ScFv-L2-aEGFRH antibody; Lane 2 is aPDL1ScFv-L2-aEGFRL;

Lane 1 of FIG. 1E is loading of PD1(m)-L1-aEGFRH antibody; Lane 2 is PD1(m)-L1-aEGFRL;

Lane 1 of FIG. 1F is loading of PD1(m)-L2-aEGFRH antibody; Lane 2 is PD1(m)-L2-aEGFRL;

Lane 1 of FIG. 1G is PD1-L3-aEGFRL; Lane 2 is aEGFR; Lane 3 is aHER2; and Lane 4 is PD1-L3-aHER2L.

FIG. 2: SEC detection of antibody fusion proteins linked by different linkers, herein A is aEGFR, B is PD1-L1-aEGFRL, C is PD1m-L1-aEGFRL, D is aPDL1ScFv-L1-aEGFRL, E is PDL1-L1-aEGFRL, F is PD1(m1)-L3-aEGFRL, G is PD1(m)-L3-aEGFRL, H is PD-L1-L3-aEGFRL, I is aPDL1ScFv-L3-aEGFRL, J is PD1-L3-aEGFRL, K is PD1-L3-aHER2L, L is PDL1-L3-aHER2L, M is PD1(m)-L3-aHER2L, N is PD1(m2)-L3-aHER2L, O is aPDL1ScFv-L3-aHER2L.

FIG. 3: Binding of different antibody fusion proteins to human EGFR

FIG. 3A shows binding of fusion protein, in which PD1 is linked to heavy chain or light chain of aEGFR by different peptide linker, to human EGFR antigen.

FIG. 3B shows binding of fusion protein, in which PD1(m) is linked to heavy chain or light chain of aEGFR by different peptide linker, to human EGFR antigen.

FIG. 3C shows binding of fusion protein, in which aPDL1 scfv is linked to heavy chain or light chain of aEGFR by different peptide linker, to human EGFR antigen.

FIG. 3D shows binding of aEGFR antibody to human EGFR antigen.

FIG. 3E shows binding of fusion protein, in which PD-1 is linked to aEGFR or aHER2 by L3 linker, to human EGFR antigen.

FIG. 4 shows binding of an aHER2 antibody fusion protein to HER2.

FIG. 5 shows binding of different antibody fusion proteins to human PD-L1.

FIG. 6 shows binding of different antibody fusion proteins to mouse PD-L1, herein isotype is an anti-RSV antibody, and PD1-L1-isotype means that PD1 is fused to an N-terminal of a light chain of the anti-RSV antibody through L1 linker.

FIG. 7 shows a stability test of an antibody fusion protein in rat plasma.

FIG. 8 shows binding of the antibody fusion protein to a cell surface antigen of a stable transgenic strain, herein A is a binding curve of an antibody fusion protein PD1-L3-aEGFRL and an MC38-EGFR stable transgenic strain, and B is a binding curve of the antibody fusion protein PD1-L3-aEGFRL and the MC38-EGFR stable transgenic strain in the presence of 500 nM EGFR-His.

FIG. 9 shows a schematic diagram of the antibody fusion protein, herein A is a schematic diagram of PD1 fused to the aEGFR antibody heavy chain through a peptide linker; and B is a schematic diagram of PD1 fused to the aEGFR antibody light chain through a peptide linker.

### DETAILED DESCRIPTION OF THE EMBODIMENTS

The present disclosure is described in detail here with reference to the following definitions and embodiments. The contents of all patents and disclosed documents mentioned herein include all sequences disclosed in these patents and disclosures, and are expressly incorporated herein by reference.

Bispecific Antibody

The “bispecific antibody” of the present disclosure is an antibody having two different antigen binding specificities. Herein the antibody has more than one specificity, and its recognized epitope may bind a single antigen or bind more than one antigen. The antibody specificity refers to the selective recognition of a specific epitope on an antigen by an antibody. A natural antibody is, for example, monospecific.

The antibody of the present disclosure is directed against two different antigens, and the two different antigens may be on the same target cell or on different target cells.

In a specific implementation mode, one target cell is a tumor cell, and the other target cell is an immune cell. The immune cell may be selected from a NK cell, a T lymphocyte or a B-cell.

In another specific implementation mode, the bispecific antibody of the present disclosure targets different antigens on the surface of the same tumor cell.

In a specific implementation mode, the present disclosure uses anti-EGFR as a scaffold, and fuses PD-1 or fragment of anti-PD-L1 at N-terminal of its heavy chain or light chain to form an antibody fusion protein which may respectively bind to EGFR and PD-L1 (as shown in FIG. 5). Such a structure not only retains pharmacokinetic properties of Fc well, but also may simultaneously target EGFR and PD-L1 ligands on the surface of tumor cells.

In a specific implementation mode, the present disclosure uses anti-HER2 as a scaffold, and fuses PD-1 or fragment of anti-PD-L1 at N-terminal of its light chain to form an antibody fusion protein which may respectively bind to HER2 and PD-L1 (as shown in FIG. 9). Such a structure not only retains the pharmacokinetic properties of the Fc well, but also may simultaneously target HER2 and PD-L1 ligands on the surface of the tumor cells.

Variable Region

As used herein, the “variable region” (light chain variable region (VL), and heavy chain variable region (VH)) refers to each pair of light chain and heavy chain domain pairs that directly participate in binding of an antibody to an antigen. The variable light chain and heavy chain regions have the same general structure and each domain contains four framework (FR) regions, sequences thereof are widely conserved, and are connected by three “hypervariable regions” (or complementarity determining regions, CDRs). The framework region adopts a β-sheet conformation and the CDR may form a loop connecting the β-sheet structure. The CDR in each chain maintains its three-dimensional structure through the framework region and forms an antigen binding site together with the CDR from the other chain. The antibody heavy chain CDR and light chain CDR regions play a particularly important role in the binding specificity/affinity of the antibody of the present disclosure.

Constant Region (Fc)

The “Fc part” of antibody does not directly participate in the binding of the antibody to the antigen, but shows a variety of effector functions. The “Fc part of antibody” is a term well known to those skilled in the art and defined based on papain digestion of the antibody. According to amino acid sequences of constant region of heavy chain, the antibodies or immunoglobulins are divided into the following categories: IgA, IgD, IgE, IgG, and IgM, and several of these may be further divided into subclasses (isotypes; it is represented that “isotypes” or “subclasses” are used interchangeably herein), such as IgG1, IgG2, IgG3 and IgG4, IgA1 and IgA2. The Fc part of the antibody directly participates in ADCC (antibody-dependent cell-mediated cytotoxicity) and CDC (complement-dependent cytotoxicity) based on complement activation, Clq binding and Fc receptor binding. Complement activation (CDC) is initiated by the binding of a complement factor C1q to the Fc part of most IgG antibody subclasses.

In one implementation scheme, the antibody of the present disclosure is characterized in that the constant chains are derived from humans. Such constant chains are well known in the prior art.

In a specific implementation mode, the Fc of the present disclosure is modified to lack effector function, namely, ADCC and/or CDC function. The loss of the effector function is achieved by at least one of the following mutations in the Fc region: E233P, L234V, L235A, ΔG236, A327G, A3305, and P331S, herein the position of the mutation is as demonstrated in SEQ ID No: 22 on the basis of EU index (Sequences of Proteins of Immunological Interest, 5-th edition, Public Health Service, National Institutes of Health, Bethesda, Md. (1991)) in Kabat, or in the corresponding position of other Fc as that of in SEQ ID No. 22. Δ means deletion, and E233P means that the 233-th amino acid is replaced from E (glutamine) to P (proline).

The “Antibody-Dependent Cell-mediated Cytotoxicity (ADCC)” refers to a cell-mediated reaction in which non-specific cytotoxic cells expressing FcR (such as Natural Killer (NK) cells, neutrophils and macrophages) recognize the antibody bound by the target cell, subsequently the lysis of the target cell is caused. Cells (NK cells) mediating ADCC only express FcγRIII, while monocytes express FcγRI, FcγRII, and FcγRIII. FcR expression on hematopoietic cells is summarized in Table 3 on Page 464 of Ravetch and Kinet, Annu. Rev. Immunol 9 (1991) 457-492.

The term “Complement-Dependent Cytotoxicity (CDC)” refers to a mechanism that induces cell death, in which the Fc effector molecule domain (one or more) of the antibody that binds to the target activates a series of enzymatic reactions, so that holes are formed in a target cell membrane. Typically, an antigen-antibody complex, such as an antigen-antibody complex on an antibody-binding target cell, binds and activates complement component C1q, and it activates complement cascade in turn, thereby the death of the target cell is caused. The activation of the complement may also cause the deposition of the complement component on the surface of the target cell, and it is beneficial to the ADCC by binding to a complement receptor (such as CR3) on a leukocyte.

The “effector function” refers to those biological activities attributable to the Fc region of the antibody, and it differs depending on the antibody isotype. Examples of the antibody effector function include: C1q binding, Complement-Dependent Cytotoxicity (CDC), Fc receptor binding, Antibody-Dependent Cell-mediated Cytotoxicity (ADCC), down-regulation of a cell surface receptor (such as a B-cell receptor), and B-cell activation.

The “deletion of the effector function” of the bispecific antibody of the present disclosure means that, compared with a control (such as an antibody with a wild-type Fc region), effector function is reduced by at least 90%, the reduction of the effector function may be detected with reference to a method disclosed in U.S. Pat. No. 8,969,526, and this article is incorporated herein by reference.

Antigen Binding Region of Antibody (CDR)

The term “antigen-binding region of an antibody” or the term “CDR” refers to the complementarity determining region in an immunoglobulin variable region. There are three CDRs in each variable region of the heavy chain and light chain, namely CDR1, CDR2, and CDR3. Exact boundaries of these CDRs are defined differently according to different systems. The systems described by Kabat (Kabat et al. (1987) and (1991)) not only provide a clear residue numbering system that may be applied to any variable regions of an antibody or a binding protein, but also provide precise residue boundaries for defining the three CDRs in each heavy chain or light chain. These CDRs may be referred to as Kabat CDRs. Chothia and colleagues (Chothia and Lesk (1987) J. Mol. Biol. 196: 901-917; Chothia et al. (1989) Nature 342: 877-883) discovered that certain subparts in Kabat CDR adopt almost the same peptide framework conformation, although there is great diversity in amino acid sequence. These subparts are named L1, L2, and L3 or H1, H2, and H3, herein “L” and “H” refer to the light chain and heavy chain, respectively. These subparts may be referred to as Chothia CDR, the boundary of which may be overlapped with the Kabat CDR. Other boundaries defining the CDRs overlapped with the Kabat CDRs are described by Padlan (1995) FASEB J. 9: 133-139 and MacCallum (1996) J. Mol. Biol. 262(5): 732-45). There are other CDR boundary definitions that may not strictly follow one of the systems in this article, but may still overlap with the Kabat CDR, although it is discovered that they may be shortened or lengthened in view of predictions or experiments that a specific residue or a residue group or even the entire CDR does not significantly affect the antigen binding. The methods used herein may utilize the CDR defined according to any of these systems, although certain implementation schemes use the CDR defined by Kabat or Chothia (CN105324396A). The “framework” or “FR” regions are those variable domain regions other than the hypervariable regions defined herein. Therefore, the light chain and heavy chain variable regions of the antibody include the domains FR1, CDR1, FR2, CDR2, FR3, CDR3, and FR4 from N-terminal to C-terminal. In particular, the CDR3 of the heavy chain is a region that most contributes to the antigen binding and defines the properties of the antibody. A term “CDR1-H” refers to a CDR1 region of the heavy chain variable region, and “CDR1-L” refers to a CDR1 region of the light chain variable region. CDR2-L, CDR3-H, etc. refers to CDR regions derived from the heavy chain (H) or the light chain (L).

The anti-EGFR antibody of the present disclosure includes CDR1-H of SEQ ID No: 33, CDR2-H of SEQ ID No: 34 and CDR3-H of SEQ ID No: 35, and CDR1-L of SEQ ID No: 36, CDR2-L of SEQ ID No: 37 and CDR3-L of SEQ ID No: 38. The anti-MUC16 antibody of the present disclosure includes CDR1-H of SEQ ID No: 49, CDR2-H of SEQ ID No: 50 and CDR3-H of SEQ ID No: 51, and CDR1-L of SEQ ID No: 52, CDR2-L of SEQ ID No: 53 and CDR3-L of SEQ ID No: 54. The anti-EGFR antibody of the present disclosure includes CDR1-H of SEQ ID No: 79, CDR2-H of SEQ ID No: 80 and CDR3-H of SEQ ID No: 81, and CDR1-L of SEQ ID No: 82, CDR2-L of SEQ ID No: 83 and CDR3-L of SEQ ID No: 84.

ScFv

A single-chain antibody variable fragment (referred to as scFv), namely a single-chain antibody, is formed by antibody heavy chain variable region (VH) and light chain variable region (VL) linked by a peptide (Linker), a molecular weight thereof is 27-30 kDa, and it is the smallest functional structural unit for all the antigen binding specificities of a parent antibody. DNA sequence of a single-chain antibody may be transformed into a mammalian cell through a viral vector or a specific mammalian expression vector. A single-chain antibody gene and other effector protein genes are fused together by a recombinant DNA technology, and after expression, a single-chain antibody fusion protein with the characteristics of the single-chain antibody and the activity of the fused effector protein may be obtained.

In a specific implementation mode, the ScFv of the anti-PD-L1 antibody of the present disclosure is selected from amino acids 1-240 of SEQ ID No. 22.

Tumor Surface Antigen

As used herein, the term “tumor surface antigen” includes a protein or a polypeptide that is preferentially expressed on the surface of the tumor cells. As used in this context, “preferentially expressed” means that the expression of antigen on the tumor cells is at least 10% higher than that on non-tumor cells (for example, 10%, 20%, 30%, 40%, 50%, 60%, 70%, 80%, 90%, 100%, 110%, 150%, 200%, 400% or higher). In certain implementation schemes, the target molecule is an antigen that is preferentially expressed on the surface of the tumor cells (such as solid tumor or hematological tumor cells): non-restrictive examples of specific tumor-associated antigens include, for example, EGFR, HER2, HER3, HER4, MUC1, MUC2, MUC3A, MUC3B, MUC4, MUC5AC, MUC5B, MUC6, MUC7, MUC8, MUC12, MUC13, MUC15, MUC16, MUC17, MUC19, MUC20, VEGFR-1 (FLT1), VEGFR-2 (KDR/FIK-1), VEGFR-3, PDGF-RA, PDGF-RB, IGF-1R, IGF2B3, K-RAS, N-RAS, Bly-S (BAFF), BAFF-R, EpCAM, SAGE, XAGE-1b, BAGE, MAGE protein (such as MAGE-1, MAGE-2, MAGE-3, MAGE-4, MAGE-6, MAGE-9, MAGE-10, and MAGE-12), GAGE-1, GAGE-2, GAGE-8, GAGE-3. GAGE-4, GAGE-5, GAGE-6, GAGE-7, XAGE-1b/GAGED2a, RAGE-1, RBAF600, CD2, CD3, CD19, CD-11a, CD16A, CD19, CD20, CD21, CD22, dipeptidyl-peptidase 4 (CD26), CD30, CD32B, CD33, CD38, CD40, CD45, CD52, CD70, CD80, CD60, CD62, CD72, CD79a, CD79B, SLAMF7 (CD139), CD123, Ly6D, Ly6E, Ly6K, gp100/Pmel17, EDAR, GFRA1 (GDNF-Ra1), MRP4, RET, STEAP1, STEAP2, TENB2, E16 (LAT1, SLC7A5), SLC35D3, MPF, SCL34A2, Sema 5b, PSCAhIg, ETBR, MSG783, FcRH1, FcRH2, NCA, MDP, IL20Ra, EphA2, EphA3, EphB2R, ASLG659, GEDA, CXCR5, P2X5, LY64, IRTA2, TMEF1, TMEM46, TMEM118, LGR5, GPR19, GPR172A, GPC3, CLL1, RNF43, KISS1R, ASPHD1, CXORF61, HAVXR1, epiregulin, amphiregulin, lipophilin, AIM-2, ALDH1A1, a-actinin-4, ARTC1, BING-4, CALCA, CASP-5, CASP-8, cdc27, CDK4, CDKN2A, CLPP, COA-1, CPSF, Cw6, RANKL, DEK-CAN, DKK1, EFTUD2, elongation factor 2, ENAH (hMena), ETV6-AML1, EZH2, FLT3-ITD, FN1, G250, MN, CAIX, GnTVf, GPNMB, HERV-K-MEL, hsp70-2, IDO1, IL13Ra2, intestinal carboxyl esterase, kallikrein 4, KIF20A, KK-LC-1, KM-HN-1, LAGE-1, LDLR-fucosyltransferase AS fusion protein, Lengsin, M-CSF, lactoglobulin-A, MART-1, Melan-A/MART-1, MART2, MCSP, mdm-2. ME-1, Meloe, MMP-2, MMP-7, mucin, MUM-1, MUM-2, MUM-3, Myosin Class I, NA88-A, PAP, neo-PAP, NFYC, NY-BR1, NY-BR62, NY-BR85, NY-ESO1, NY-ESO-1/LAGE-2, RAB38/NY-MEL-1, OA1, OGT, OS-9, p53, PAX3, PAX5, PBF, PML-RARa, PRAME, PRDX5, PSMA(FOLH1), PTPRK, RGS5, Rho, RhoC, RNF43, RU2AS, protein isolate 1, SIRT2, SNRPD1, SOX10, Sp17, SSX-2, SSX-4, survivin, SYT-SSX1 or -SSX2, TAG-1, TAG-2, telomerase, TGF-ρ, TGF-beta RII, TRAG-3, triose phosphate isomerase, TRP-2, TRP2-INT2, VEGF, WT1, TRPM4, CRIPTO, glycoprotein IIb/IIIa receptor, glycolipid GD2, GD3, CCR4, CCR5, folate receptor 1 (FOLR1), IFNγ, IFNα, β, ω receptor1, TROP-2, Glyco-protein NMB, MMP9, GM3, mesothelin, fibronectin extra-domain B, endoglin, Rhesus D, plasma kallikrein, CS, thymic stromal lymphopoietin, mucosal addressin cell adhesion molecule, nectin 4, NGcGM3, DLL3, DLL4, CL EC12A, KLB, FGFR1C, CEA, BCMA, p-cadherin, FAP, DR1, DR5, DR13, PLK, B7-H3, c-Met, gpA33, gp100/Pme117, gp100, TRP-1/gp75, BCR-ABL, AFP, ALK, β-chain protein, BRCA1, BORIS, CA9, Caspase-8, CDK4, CTLA4, Cyclin-B1, Cyclin D1, Cyclin-A1, CYP1B1, Fra-1, GloboH, Glypican-3, GM3, HLA/B-RAF, hTERT, LMP2, mesothelin, ML-IAP, NA17, OX40, p15, PPLR, PCTA-1, PLAC1, PRLR, PRAME, SART-1, SART-3, TAG-72, TMPRSS2, Tn, tyrosinase and urinary plaque protein-3.

Immune Checkpoint Protein

Immune checkpoint is a type of signals that regulate T-cell receptor (TCR) antigen recognition during an immune response, which includes a co-stimulatory immune signal that stimulates immunity and a co-suppressive immune signal that suppresses the immunity. The immune checkpoint may prevent autoimmune damage caused by excessive activation of immune cells (for example, a T-cell). The tumor cells use the human immune system, namely a protective mechanism, to overexpress immune checkpoint proteins, thereby an anti-tumor response of the human immune system is inhibited and immune escape is formed. Immune checkpoint therapy uses a co-stimulatory signal agonist or a co-inhibitory signal antagonist to make the immune system work normally. Common immune checkpoint proteins include CD27, CD28, CD40, CD122, CD137, OX40, GITR, ICOS, A2AR, B7-H3, B7-H4, BTLA, CD40, CTLA-4, IDO, KIR, LAGS, PD-1, PD-L1, PD-L2, TIM-3, VISTA, CEACAM1, GARP, PS, CSF1R, CD94/NKG2A, TDO, GITR, TNFR and FasR/DcR.

The immune checkpoint proteins are mainly expressed on the surface of the immune cells. The immune checkpoint proteins are also expressed on the surface of the tumor cells. For example, PD-L1 is highly expressed on the surface of many tumor cells, such as lung cancer, breast cancer, malignant melanoma, esophageal cancer, gastric cancer, and pancreatic cancer.

Immune Cell

The immune cell described in this article refers to a cell that may recognize the antigen and induce the specific immune response. Immune cells include but not limited to the T-cell, the B-cell, the natural killer cell (NK) and the like.

PD-1

PD-1, namely programmed cell death factor 1, is a co-stimulatory molecule belonging to CD28 family. It is inducibly expressed on the surface of activated T-cell, B-cell and NK cell. The interaction with its ligands plays an important role in autoimmunity, transplantation immunity, tumor immunity and chronic viral infection.

PD-1 has two specific ligands, namely PD-L1 and PD-L2. In a gene level, PD-L2 has 37.4% of homology with PD-L1. PD-L1 is expressed on T-cells, B-cells, dendritic cells, macrophages, mesenchymal stem cells, and some non-hematopoietic cells (including cardiovascular endothelial cells, renal tubular epithelial cells, glial cells, pancreatic β cells, and liver cells, etc.), while PD-L2 is mainly expressed on endritic cells, monocytes, mast cells derived from bone marrow, and B-cells in germinal center. In humans, PD-L2 is also slightly expressed on vascular endothelial and T-cell. After binding with PD-L1/PD-L2, PD-1 may inhibit the activation of initial T-cells and the function of effector T-cells, induce the production of regulatory T-cells and maintain its inhibitory function.

The PD-1 used in the present disclosure is mammalian-derived PD-1, for example, human-derived PD-1, and mouse-derived PD-1. Preferably, the present disclosure uses the human-derived PD-1, and it may have amino acids 1-143 of SEQ ID No: 6, SEQ ID No. 14, and SEQ ID No. 44. Mammalian-derived PD-1 protein molecules have a high degree of identity.

EGFR

Epidermal growth factor receptor (EGFR, ErbB1 or HER1 for short) is a membrane glycoprotein derived from proto-oncogene C-erbB1 EGFR is mainly expressed on epithelial cells with an extracellular region, a transmembrane region and an intracellular region. It is showed that abnormal high EGFR expression in many malignant tumors is observed, and it is often related to cancer cell proliferation, neoangiogenesis, tumor metastasis, and inhibition of cancer cell apoptosis (anti-apoptosis). Possible mechanisms thereof are as follows: EGFR overexpression activates and enhances downstream signal transduction; the mutant EGFR might also enhance the downstream signal transduction; continuous activation of EGFR is caused by overexpression of an EGFR ligand; it is also possible that an autocrine function is enhanced; down-regulation mechanism of EGFR is destroyed; and an abnormal signal transduction pathway is activated, etc.

Pharmaceutical Composition

The pharmaceutical composition as described herein is prepared by mixing the bispecific antibody of the present disclosure with the desired purity and one or more optional pharmaceutically acceptable carriers. It could be freeze-drying preparation or aqueous solution. The pharmaceutically acceptable carrier is generally non-toxic to a recipient at dosage and concentration used.

The bispecific antibody of the present disclosure may be administered as a single active ingredient, or in combination with, for example, an adjuvant or with other drugs such as immunosuppressive or immunoregulatory agents or other anti-inflammatory agents, for the treatment or prevention of diseases, for example, acute lymphoblastic leukemia (ALL), acute medullary leukemia (AML), adrenal cortical cancer, anal cancer, appendix cancer, astrocytoma, basal cell carcinoma, brain tumor, cholangiocarcinoma, bladder cancer, bone cancer, breast cancer, bronchial tumor, Burkitt lymphoma, cancer of unknown primary origin, heart tumor, cervical cancer, chordoma, chronic lymphocytic leukemia (CLL), chronic myelogenous leukemia (CML), chronic myeloproliferative neoplasms, colon cancer, colorectal cancer, craniopharyngioma, skin T-cell lymphoma, ductal carcinoma, embryonal tumor, endometrial cancer, ependymoma, esophageal cancer, nasal cavity glioma, fibrous histiocytoma, Ewing's sarcoma, eye cancer, Germ cell tumor, gallbladder cancer, gastric cancer, gastrointestinal carcinoid tumor, gastrointestinal stromal tumor, gestational trophoblastic disease, glioma, head and neck cancer, hairy cell leukemia, hepatocellular carcinoma, histiocytosis, Hodgkin lymphoma, hypopharyngeal cancer, intraocular melanoma, islet cell tumor, Kaposi's sarcoma, kidney cancer, Langerhans cell histiocytosis, laryngeal cancer, leukemia, lip and oral cavity cancer, liver cancer, lobular carcinoma in situ, lung cancer, lymphoma, macroglobulinemia, malignant fibrous histiocytoma, melanoma, Merkel cell carcinoma, mesothelioma, occult primary metastatic squamous neck cancer, midline tract cancer involving a NUT gene, oral cancer, multiple endocrine neoplasia syndrome, multiple myeloma, granuloma fungoides, myelodysplastic syndrome, myelodysplastic/myelodysplastic neoplasms, nasal cavity and paranasal sinus cancer, nasopharyngeal carcinoma, neuroblastoma, non-Hodgkin's lymphoma, non-small cell lung cancer, oropharyngeal cancer, osteosarcoma, ovarian cancer, pancreatic cancer, papillomatosis, paraganglioma, parathyroid cancer, penile cancer, pharynx cancer, pheochromocytoma, pituitary tumor, pleuropulmonary blastoma, primary central nervous system lymphoma, prostate cancer, rectal cancer, renal cell carcinoma, renal pelvis and ureter cancer, retinoblastoma, rhabdomyomas, salivary gland cancer, Sezary syndrome, skin cancer, small cell lung cancer, small intestine cancer, soft tissue sarcoma, spinal cord tumor, gastric cancer, T-cell lymphoma, teratoma, testicular cancer, throat cancer, thymoma and thymic cancer, thyroid cancer, urethral carcinoma, uterus cancer, vagina cancer, vulva cancer, and Wilms tumor.

### EMBODIMENT

Embodiments are merely illustrative, and are not intended to limit the present disclosure in any manners.

The meanings of the abbreviations are as follows: “h” refers to hours, “min” refers to minutes, “s” refers to seconds, “ms” refers to milliseconds, “d” refers to days, “μL” refers to microliters, “mL” refers to milliliters, and “L” refers to liters, “bp” refers to base pairs, “mM” refers to millimoles, “μM” refers to micromoles, and “nM” refers to nanomoles.

### Embodiment 1: Construction of Eukaryotic Expression Vector of Antibody Fusion Protein

A heavy chain variable region of an anti-human EGFR antibody (a EGFR VH) (1-357 bp of SEQ ID No.1), a light chain variable region of an anti-human EGFR antibody (aEGFR VL) (1-321 bp of SEQ ID NO.3), a heavy chain variable region of an anti-human HER2 antibody (aHER2 VH) (1-360 by of SEQ ID No. 59), a light chain variable region of an anti-human HER2 antibody (aHER2 VL) (1-321 bp of SEQ ID NO. 57), a human PD-1 gene (PD1) (1-429 bp of SEQ ID No.5), a human PD-1 gene mutant (PD1(m)) (1-429 bp of SEQ ID No.13), a human PD-1 gene mutant (PD1(m1)) (1-429 bp of SEQ ID No. 43), an scfv fragment of an anti-PD-L1 antibody (aPDL1 scfv) (1-720 bp of SEQ ID No. 21) (the above genes are all synthesized by IDT. Inc) are amplified by PCR. The amplified aEGFR VH and aEGFR VL genes are respectively cloned into a pFuse-hIgG1-Fc2 vector (InvivoGen) (herein hIgG1-Fc on the vector contains 9 mutations: E233P, L234V, L235A, ΔG236, A327G, A3305, P331S, E356D, and M358L which are all completed by our laboratory) and a pFuse2-CLIg-Hk vector (InvivoGen) by a method of enzyme digestion and linkage. The amplified PD1, PD1(m), and aPDL1 scfv genes are cloned into N-terminals of aEGFRVH and aEGFRVL of pFuse-aEGFR HC and/or pFuse-aEGFR LC constructed above through a peptide linker (L1 or L2) by the method of enzyme digestion and linkage, or PD1, PD1(m), PD1 (m1), and aPDL1 scfv are cloned to an N-terminal of aHER2 antibody or aEGFR antibody light chain by a peptide linker L3. All constructed vectors are verified by sequencing.

TABLE 1
Sequence name

| | | Nucleic acid | Amino acid |
|---|---|---|---|
| Construct | Description | sequence No. | sequence No. |
| aEGFR HC | Anti-human EGFR antibody | 1 | 2 |
| | heavy chain | | |
| aEGFR LC | Anti-human EGFR antibody | 3 | 4 |
| | light chain | | |
| PD1-L1- aEGFR HC | PD1 is fused to an N-terminal of | 5 | 6 |
| | the anti-human EGFR antibody | | |
| | heavy chain via L1 | | |
| PD1-L2-aEGFR HC | PD1 is fused to an N-terminal of | 7 | 8 |
| | the anti-human EGFR antibody | | |
| | heavy chain via L2 | | |
| PD1-L1- aEGFR LC | PD1 is fused to an N-terminal of | 9 | 10 |
| | the anti-human EGFR antibody | | |
| | light chain via L1 | | |
| PD1-L2- aEGFR LC | PD1 is fused to an N-terminal of | 11 | 12 |
| | the anti-human EGFR antibody | | |
| | light chain via L2 | | |
| PD1(m)-L1-aEGFR HC | PD1(m) is fused to an N-terminal | 13 | 14 |
| | of the anti-human EGFR antibody | | |
| | heavy chain via L1 | | |
| PD1(m)-L2-aEGFR HC | PD1(m) is fused to an N-terminal | 15 | 16 |
| | of the anti-human EGFR antibody | | |
| | heavy chain via L2 | | |
| PD1(m)-L1- aEGFR LC | PD1(m) is fused to an N-terminal | 17 | 18 |
| | of the anti-human EGFR antibody | | |
| | light chain via L1 | | |
| PD1(m)-L2-aEGFR LC | PD1(m) is fused to an N-terminal | 19 | 20 |
| | of the anti-human EGFR antibody | | |
| | light chain via L2 | | |
| aPDL1 ScFv-L1-aEGFR HC | aPDL1 ScFv is fused to an | 21 | 22 |
| | N-terminal of the anti-human | | |
| | EGFR antibody heavy chain via L1 | | |
| aPDL1 ScFv-L2-aEGFR HC | aPDL1 ScFv is fused to an | 23 | 24 |
| | N-terminal of the anti-human | | |
| | EGFR antibody heavy chain via L2 | | |
| aPDL1 ScFv-L1-aEGFR LC | aPDL1 ScFv is fused to an | 25 | 26 |
| | N-terminal of the anti-human | | |
| | EGFR antibody light chain via L1 | | |
| aPDL1 ScFv-L2-aEGFR LC | aPDL1 ScFv is fused to an | 27 | 28 |
| | N-terminal of the anti-human | | |
| | EGFR antibody light chain via L2 | | |
| L1 | peptide linker | 29 | 30 |
| L2 | peptide linker | 31 | 32 |
| aEGFR CDR1-H | CDR1 region of aEGFR heavy | / | 33 |
| | chain variable region | | |
| aEGFR CDR2-H | CDR2 region of aEGFR heavy | / | 34 |
| | chain variable region | | |
| aEGFR CDR3-H | CDR3 region of aEGFR heavy | / | 35 |
| | chain variable region | | |
| aEGFR CDR1-L | CDR1 region of aEGFR light | / | 36 |
| | chain variable region | | |
| aEGFR CDR2-L | CDR2 region of aEGFR light | / | 37 |
| | chain variable region | | |
| aEGFR CDR3-L | CDR3 region of aEGFR light | / | 38 |
| | chain variable region | | |
| aMUC16 HC | Heavy chain of anti-MUC16 | 39 | 40 |
| | antibody | | |
| aMUC16 LC | Light chain of anti-MUC16 | 41 | 42 |
| | antibody | | |
| PD1(m1)-L1-aEGFR LC | PD1 (m1) is fused to an | 43 | 44 |
| | N-terminal of the anti-human | | |
| | EGFR antibody light chain via L1 | | |
| PD1-L1-aMUC16 LC | PD1 is fused to an N-terminal of | 45 | 46 |
| | the anti-human MUC16 antibody | | |
| | light chain via L1 | | |
| PD1(m)-L1-aMUC16 LC | PD1(m) is fused to an N-terminal | 47 | 48 |
| | of the anti-human MUC16 | | |
| | antibody light chain via L1 | | |
| aMUC16 CDR1-H | CDR1 region of aMUC16 heavy | / | 49 |
| | chain variable region | | |
| aMUC16 CDR2-H | CDR2 region of aMUC16 heavy | / | 50 |
| | chain variable region | | |
| aMUC16 CDR3-H | CDR3 region of aMUC16 heavy | / | 51 |
| | chain variable region | | |
| aMUC16 CDR1-L | CDR1 region of aMUC16 light | / | 52 |
| | chain variable region | | |
| aMUC16 CDR2-L | CDR2 region of aMUC16 light | / | 53 |
| | chain variable region | | |
| aMUC16 CDR3-L | CDR3 region of aMUC16 light | / | 54 |
| | chain variable region | | |
| PD1-L3-aEGFR-LC | PD1 is fused to an N-terminal of | 55 | 56 |
| | the anti-human EGFR antibody | | |
| | light chain via L3 | | |
| aHER2 LC | Anti-human HER2 antibody light | 57 | 58 |
| | chain | | |
| aHER2 HC | Anti-human HER2 antibody heavy | 59 | 60 |
| | chain | | |
| PD1(m)-L3-aEGFR-LC | PD1(m) is fused to an N-terminal | 61 | 62 |
| | of the anti-human EGFR antibody | | |
| | light chain via L3 | | |
| aPDL1-L3-aEGFR-LC | aPDL1 scfv is fused to an | 63 | 64 |
| | N-terminal of the anti-human | | |
| | EGFR antibody light chain via L3 | | |
| PDL1-L3-aEGFR-LC | PDL1 is fused to an N-terminal of | 65 | 66 |
| | the anti-human EGFR antibody | | |
| | light chain via L3 | | |
| PD1(m1)-L3-aEGFR-LC | PD1 (m1) is fused to an | 67 | 68 |
| | N-terminal of the anti-human | | |
| | EGFR antibody light chain via L3 | | |
| PD1-L3-aHER2-LC | PD1 is fused to an N-terminal of | 69 | 70 |
| | the anti-human HER2 antibody | | |
| | light chain via L3 | | |
| PD1(m)-L3-aHER2-LC | PD1(m) is fused to an N-terminal | 71 | 72 |
| | of the anti-human HER2 antibody | | |
| | light chain via L3 | | |
| aPDL1-L3-aHER2-LC | aPDL1 scfv is fused to an | 73 | 74 |
| | N-terminal of the anti-human | | |
| | HER2 antibody light chain via L3 | | |
| PDL1-L3-aHER2-LC | PDL1 is fused to an N-terminal of | 75 | 76 |
| | the anti-human HER2 antibody | | |
| | light chain via L3 | | |
| PD1(m1)-L3-aHER2-LC | PD1 (m1) is fused to an | 77 | 78 |
| | N-terminal of the anti-human | | |
| | HER2 antibody light chain via L3 | | |
| aHER2 CDR1-H | CDR1 region of aHER2 heavy | / | 79 |
| | chain variable region | | |
| aHER2 CDR2-H | CDR2 region of aHER2 heavy | / | 80 |
| | chain variable region | | |
| aHER2 CDR3-H | CDR3 region of aHER2 heavy | / | 81 |
| | chain variable region | | |
| aHER2 CDR1-L | CDR1 region of aHER2 light | / | 82 |
| | chain variable region | | |
| aHER2 CDR2-L | CDR2 region of aHER2 light | / | 83 |
| | chain variable region | | |
| aHER2 CDR3-L | CDR3 region of aHER2 light | / | 84 |
| | chain variable region | | |
| L3 | peptide linker | / | 85 |

Note:
PD1: Human PD-1
PD1(m): Human PD-1 mutant
PD1(m1): Human PD-1 mutant
LC: Antibody light chain
HC: Antibody heavy chain

### Embodiment 2: Expression, Purification and SEC(Size Exclusion Chromatography) Detection of Antibody Fusion Protein

The heavy chain and light chain of the fusion protein expression vector constructed in Embodiment 1 are transiently transfected into FreeStyle HEK293 cells (ThermoFisher), and the molar ration of the heavy chain and light chain used during transfection is 1:1:28 ml of FreeStyle HEK 293 (3×10⁷ cells/nil) is cultured in 125 ml of a cell culture flask, the plasmids are diluted with 1 ml of Opti-MEM (Invitrogen) and added to 1 ml of Opti-MEM containing 60 μl of 293Fectin (Invitrogen). After incubation at room temperature for 30 min, the plasmid-293Fectin mixture is added to cell culture and then incubated at 125 rpm, 37° C., and 5% CO₂. Cell culture supernatant is collected in 96 h after transfection, purified by Protein A Resin (Genscript), and detected by SDS-PAGE. SDS-PAGE diagrams are shown in FIG. 1, which indicated that the antibody fusion protein is successfully expressed.

The obtained antibody fusion protein purified by Protein A resin is analyzed through by SEC by GE AKTA. The chromatography column used is: a Superdex 200 Increase 10/300GL gel exclusion chromatography column, solution used for gel exclusion chromatography is PBS buffer (0.010M phosphate buffer, 0.0027M KCl, 0.14M NaCl, pH7.4). It is seen from a chromatogram in FIG. 2 that, the expression of antibody fusion proteins linked by different linkers has considerable purity.

### Embodiment 3: Mass Spectrometry (MS) Analysis

A sample purified by Protein A resin and obtained in Embodiment 2 is incubated with PNGase F (NEB) at 37° C. for 8 hours, and treated with 10 mM dithiothreitol, the sample is injected into a 3005B-C8, 2.1×50 mm column of HPLC-Q-TOF-MS (Agilent, USA), and mass spectrometry analysis is performed. As shown in Table 2, molecular weights of the antibody fusion proteins in different fusion forms detected by mass spectrometry are basically consistent with theoretical prediction values.

TABLE 2
Mass spectrometry analysis

| | Heavy chain | | Light chain | |
|---|---|---|---|---|
| | Theoretical | Detection | Theoretical | Detection |
| | molecular | molecular | molecular | molecular |
| Antibody | weight (D) | weight (D) | weight (D) | weight (D) |
| PD1-L1-aEGFRH | 66463.7 | 68720.61 | 23357.94 | 23354.63 |
| PD1-L1-aEGFRL | 48761.99 | 48739.1 | 41059.65 | 43319.41 |
| PD1-L2-aEGFRH | 68200.65 | 70458.78 | 23357.94 | 23354.55 |
| PD1-L2-aEGFRL | 48761.99 | 48739.1 | 42796.6 | 45056.22 |
| aPDL1 ScFv-L1-aEGFRH | 75938.05 | 75929.48 | 23357.94 | 23354.71 |
| aPDL1 ScFv-L1-aEGFRL | 48761.99 | 48739.1 | 50549.03 | 50526.94 |
| aPDL1 ScFv-L2-aEGFRH | 77675 | 77667.47 | 23357.94 | 23354.55 |
| aPDL1 ScFv-L2-aEGFRL | 48761.99 | 48739.15 | 52270.95 | 52264.34 |
| PD1(m)-L1-aEGFRH | 66416.58 | 68670.77 | 23357.94 | 23354.66 |
| PD1(m)-L1-aEGFRL | 48761.99 | 48738.87 | 41012.53 | 43272.94 |
| PD1(m)-L2-aEGFRH | 68153.53 | 70412.89 | 23357.94 | 23354.63 |
| PD1(m)-L2-aEGFRL | 48761.99 | 48739.02 | 42749.48 | 45030.97 |

Note:
PD1-L1-aEGFRH: Antibody of which a PD1 protein is fused to an N-terminal of an aEGFR antibody heavy chain
PD1-L1-aEGFRL: Antibody of which a PD1 protein is fused to an N-terminal of an aEGFR antibody light chain

### Embodiment 4: Function Detection of Anti-EGFR Fusion Protein

4.1. Binding Human EGFR ELISA Detection

hEGFR-hIGg1Fc (SinoBiological) (100 ng/well) is coated in a 96-well plate, and incubated overnight at 4° C. After blocking with PBST containing 2% skimmed milk powder (0.5% Tween-20 in PBS) for 1 hour at room temperature, gradient-diluted (10 pM-1.2 nM) antibody fusion proteins are added and incubated at room temperature for 2 h. After washing for 4-5 times with PBST containing 2% skimmed milk powder, an anti-human kappa light chain (Sigma A7146, 1:3000) secondary antibody is added, incubated at room temperature for 1h and washed with PBST containing 2% skimmed milk powder for 4-5 times. Color development is performed with a QuantaBlu fluorescent peroxidase substrate (Life technologies, Cat.15169) (readings performed at 325 nm and 420 nm), or by using a TMB color reagent (BioLegend, Cat.421101) (readings performed at 650 nm). Data was analyzed by nonlinear regression using specific binding model in Prizm Graphpad software. Results are shown in FIG. 3, the scaffold from different antibody fusion proteins (PD-1-aEGFR fusion, PD1(m)-aEGFR fusion, PD1(m1)-aEGFR fusion or aPDL1 scfv-aEGFR fusion) has higher affinity with EGFR, which is basically the same as that of anti-EGFR IgG with EGFR in FIG. 3D.

4.2. Human HER2 Binding ELISA Detection

hHER2-His (Acro) (100 ng/well) is coated in a 96-well plate and incubated overnight at 4° C. After blocking with PBST containing 2% skimmed milk powder (0.5% Tween-20 in PBS) for 1 hour at room temperature, gradient-diluted (10 pM-1.2 nM) antibody fusion proteins PD1-L3-aEGFRL, PD1(m)-L3-aHER2L, or PD1(m1)-L3-aHER2L are added and incubated at room temperature for 2h. After washing for 4-5 times with PBST containing 2% skimmed milk powder, an anti-human kappa light chain (Sigma A7146, 1:3000) secondary antibody is added and incubated for 1 h at room temperature. After washing for 4-5 times with PBST containing 2% skimmed milk powder, color development is performed with a QuantaBlu fluorescent peroxidase substrate (Life technologies, Cat.15169) (readings performed at 325 nm and 420 nm). Data was analyzed by nonlinear regression using specific binding model in Prizm Graphpad software.

Results are shown in FIG. 4, the fusion of PD1 or its mutants to aHER2 does not affect the binding of anti-HER2 antibody to HER2 in the fusion protein.

4.3. Binding Human PD-L1 or Mouse PD-L1 ELISA Detection

hPD-L1-hIGg1 Fc (SinoBiological) or mouse PD-L1-Fc (SinoBiological) (100 ng/well) is coated in 96-well plates, and incubated overnight at 4° C. After blocking with PBST containing 2% skimmed milk powder (0.5% Tween-20 in PBS) at room temperature for 1 h, gradient-diluted (25 pM-3 nM) antibody fusion proteins are added and incubated for 2h at room temperature. After washing for 4-5 times with PBST containing 2% skimmed milk powder, an anti-human kappa light chain (Sigma A7146, 1:3000) secondary antibody is added and incubated at room temperature for 1 h. After washing with PBST containing 2% skimmed milk powder for 4-5 times, color development is performed with QuantaBlu fluorogenic peroxidase substrate (Life technologies, Cat. 15169) (readings performed at 325 nm and 420 nm). Data was analyzed by nonlinear regression using specific binding model in Prizm Graphpad software.

Results of the binding with human PD-L1 are shown in FIG. 5, the fusion of PD-1, PD1(m), PD1(m) or aPDL1 scfv to the antibody scaffold (such as C-terminal of the anti-EGFR or anti-HER2 heavy chain or light chain) does not affect its binding to PD-L1 (FIG. 5A-5E). The fusion is similar to this.

Binding to mouse PD-L1 showed similar results (see FIG. 6). PD1, PD1(m), PD1(m1) or aPDL1 scfv in different fusion proteins could bind to mouse PD-L1, and the antibody scaffold has little effect on the binding.

4.4. Plasma Stability Test

The bispecific antibody or control is added to a tube containing 100 μl of freshly separated rat serum (final concentration 1 μM), and incubated at 37° C. for different times (such as 0 h, 5 min, 15 min, 30 min, 1 h, 3 h, 6 h, 24 h, 48 h and 72 h). Incubated samples are quickly frozen with liquid nitrogen and placed at −80° C. for further use. The content of the antibody in each tube is detected by coated PD-L1 in sandwich ELISA. The detailed detection process is as described in Embodiment 4.3.

Results are shown in FIG. 7, the antibody fusion protein is relatively stable in rat serum.

4.5. Antibody Fusion Protein Binding to Cell Surface PD-L1 and/or EGFR

MC38 cells (MC38-EGFR, constructed by our laboratory) that highly express EGFR (DMEM medium containing 10% FBS, and 1% double antibody) are cultured. After trypsinization, 2×104/well MC38-EGFR cells are placed on a 96-well flat-bottomed black plate and incubated overnight at 37° C. with 5% CO₂. After washing for 3 times with PBS, supernatant is discarded by centrifuging and 8% formalin solution is added and incubated at a room temperature for 15 min. After discarding the formalin solution, the antibody fusion proteins with different concentrations are directly added for cell binding analysis, or the antibody fusion proteins with different concentrations are added in the presence of 500 nM EGFR-His for competitive binding analysis. The unbound antibody fusion protein is washed with PBS containing 2% FBS, a secondary antibody Mouse Anti-Human IgG Fc-APC (southern biotech) is added, and incubated at 4° C. for 1 hour. After washing for three times with PBS of 2% FBS, the fluorescence intensity is detected by a flow cytometer.

Results are shown in FIG. 8 and Table 3, the binding (A) of PD1-L3-aEGFRL to MC38-EGFR cells may be competitively inhibited (B) by free EGFR-his in the solution.

TABLE 3
Binding of PD1-L3-aEGFR and MC38-EGFR cells

| EC₅₀(nM) | PD1-L3-aEGFRL | aEGFR |
|---|---|---|
| Direct binding | 0.6 | 0.3 |
| Competitive binding | 82.3 | 121.5 |
| (In the presence of 500 nM EGFR-His) | | |

What is claimed is:

1. A bispecific antibody, comprising: a first binding domain which targets a surface antigen of a first target cell, and a second binding domain which binds to an immune checkpoint protein on the surface of a second target cell, wherein the first binding domain is an antibody comprising a constant region, a heavy chain variable region, and a light chain variable region, the second binding domain is linked to N-terminal of the heavy chain variable region or the light chain variable region of the first binding domain, wherein the first target cell is a tumor cell, the second target cell is the same cell as the first target cell, or the second target cell is an immune cell.

2. The bispecific antibody according to claim 1, wherein the antibody targets two different antigens on the same tumor cell.

3. The bispecific antibody according to claim 1, wherein the antibody targets two different antigens on the tumor cell and the immune cell.

4. The bispecific antibody according to claim 1, wherein the immune cell is selected from an NK cell, a T lymphocyte and a B-cell;
preferably, the tumor cell surface antigen is selected from one of a growth factor receptor family, a receptor tyrosine kinase family or a mucin family;
preferably, the growth factor receptor is selected from one of an epidermal growth factor receptor family, a tyrosine kinase receptor family, a vascular endothelial growth factor receptor family, an insulin-like growth factor 1 receptor and a platelet-derived growth factor receptor family;
preferably, the growth factor receptor is selected from an epidermal growth factor receptor (EGFR), a vascular endothelial growth factor receptor 1 (VEGFR-1, FLT1), a vascular endothelial growth factor receptor 2 (VEGFR-2, KDR/Flk-1), a vascular endothelial growth factor receptor 3 (VEGFR-3), an insulin-like growth factor 1 receptor (IGF-1R), a platelet-derived growth factor receptor A subunit (PDGF-RA) and a platelet-derived growth factor receptor B subunit (PDGF-RB);
preferably, the receptor tyrosine kinase is selected from one of an ERBB2 receptor tyrosine kinase 2 (HER2), an ERBB2 receptor tyrosine kinase 3 (HER3) and an ERBB2 receptor tyrosine kinase 4 (HER4);
preferably, the mucin family is selected from one of mucin1 (MUC1), MUC2, MUC3A, MUC3B, MUC4, MUC5AC, MUC5B, MUC6, MUC7, MUCK, MUC12, MUC13, MUC15, MUC16, MUC17, MUC19 and MUC20.

5. (canceled)

6. (canceled)

7. (canceled)

8. (canceled)

9. (canceled)

10. The bispecific antibody according to claim 1, wherein the immune checkpoint protein is one selected from PD-1, PD-L1, CTLA-4, LAG-3, OX40, CD28, CD40, CD47, CD70, CD80, CD122, GTIR, A2AR, B7-H3 (CD276), B7-H4, IDO, KIR, Tim-3 and 4-1BB (CD137).

11. The bispecific antibody according to claim 1, wherein the antibody targets EGFR and PD-L1, or targets MUC16 and PD-L1, or targets HER2 and PD-L1.

12. The bispecific antibody according to claim 1, wherein the second binding domain is PD1.

13. The bispecific antibody according to claim 1, wherein the second binding domain is human PD1 or a variant thereof.

14. The bispecific antibody according to claim 1, wherein the second binding domain is ScFv of an anti-PD-L1 antibody or a fragment thereof.

15. The bispecific antibody according to claim 1, wherein the first binding domain only has a function of binding to a cell surface antigen, or has both Fc effector function and function of binding to a cell surface antigen.

16. The bispecific antibody according to claim 15, wherein the second binding domain is selected from amino acids 1-143 of SEQ ID NO: 6, amino acids 1-143 of SEQ ID NO: 14, amino acids 1-143 of SEQ ID NO: 44 or amino acids 1-240 of SEQ ID No: 22.

17. The bispecific antibody according to claim 1, wherein the heavy chain variable region of the first binding domain comprises the region selected from the followings: CDR1-H, CDR2-H and CDR3-H, and the light chain variable region comprises the region selected from the followings CDR1-L, CDR2-L and CDR3-L;
a) CDR1-H shown in SEQ ID No: 33, CDR2-H shown in SEQ ID No: 34 and CDR3-H shown in SEQ ID No: 35; CDR1-L shown in SEQ ID No: 36, CDR2-L shown in SEQ ID No: 37, and CDR3-L shown in SEQ ID No: 38;
b) CDR1-H shown in SEQ ID No. 49, CDR2-H shown in SEQ ID No: 50, and CDR3-H shown in SEQ ID No: 51; and CDR1-L shown in SEQ ID No: 52, CDR2-L shown in SEQ ID No: 52, and CDR3-L shown in SEQ ID No: 53; or
c) CDR1-H shown in SEQ ID No. 79, CDR2-H shown in SEQ ID No: 80, and CDR3-H shown in SEQ ID No: 81; and CDR1-L shown in SEQ ID No: 82, CDR2-L shown in SEQ ID No: 83, and CDR3-L shown in SEQ ID No: 84.

18. The bispecific antibody according to claim 1, wherein the second binding domain is linked to an N-terminal of the heavy chain variable region or the light chain variable region of the first binding domain, and is linked by a peptide linker.

19. The bispecific antibody according to claim 18, wherein the peptide linker has the amino acids set forth in L1 of SEQ ID No: 30, L2 of SEQ ID No: 32, or L3 of SEQ ID No: 85.

20. The bispecific antibody according to claim 1, wherein Fc region of the first binding domain is selected from amino acids 223-448 of SEQ ID No: 2.

21. A nucleic acid encoding the bispecific antibody according to claim 1.

22. An expression vector comprising the nucleic acid according to claim 21.

23. A host cell, wherein it comprises the expression vector according to claim 22.

24. A pharmaceutical composition, wherein it comprises the bispecific antibody according to claim 1.

25. A method for treating autoimmune disease or cancer comprising: providing the antibody according to claim 1, administering a corresponding effective dose of the bispecific antibody to a patient with autoimmune disease or cancer.
